(19) United States
(12) Patent Application Publication
(10) Pub. No.: US 2025/0164511 A1
(43) Pub. Date: May 22, 2025

(54) Diazobutanone Linker - Assisted High-Throughput Quantitative Analysis for Phosphate and Sulfate Containing Lipids

(71) Applicant: Wisconsin Alumni Research Foundation, Madison, WI (US)

(72) Inventors: Lingjun Li, Madison, WI (US); Ting-Jia Gu, Madison, WI (US); Peng-Kai Liu, Madison, WI (US)

(21) Appl. No.: 18/518,234

(22) Filed: Nov. 22, 2023

(51) Int. Cl. G01N 33/92; G01N 33/58

(52) U.S. Cl. CPC G01N 33/92; G01N 33/582; G01N 2458/15

(57) ABSTRACT

A linker comprising both a diazo group and a ketone group capable of directly conjugating phosphodiester and sulfate groups on biomolecules using the diazo group, thereby enabling the functionalization of phosphate and sulfate-containing biomolecules via oxime bond formation. The linker is compact and volatile, facilitating easy cleanup by vacuum, which in turn minimizes sample loss and improves experimental operations. The linker exhibits high derivatization efficiency and chemoselectivity, accommodating a variety of functional groups on the biomolecules. By coupling the linker with isobaric mass tags, multiplexed quantitative analysis can be achieved for an extensive variety of lipid classes.

### STATEMENT REGARDING FEDERALLY SPONSORED RESEARCH OR DEVELOPMENT

This invention was made with government support under AG052324 and AG078794 awarded by the National Institutes of Health. The government has certain rights in the invention.

### BACKGROUND OF THE INVENTION

Lipidomics is a rapidly expanding field that aims to characterize the lipidome in biological systems to elucidate lipid functions and their roles in disease pathogenesis¹. Phospholipids (PL) and glycolipids, the major components of lipidomes, regulate cell membrane dynamics, serve as storage depots of energy, and are precursors of bioactive metabolite^{s2-4}. The regulation is determined by lipid chemical structures and relative abundance between each⁵. Recently, the associations between lipidomes and diseases have stimulated the development of quantitative lipidomics for discovering disease biomarkers and therapeutic targets⁶⁻⁸. However, high-throughput approaches for quantifying lipidomes lag behind genomics and proteomics mainly due to the high diversity in chemical structures and physiochemical properties of lipids. Therefore, a high-throughput strategy that can accurately identify and quantify a broad range of lipids is crucial for addressing critical biological questions relevant to lipidomes and lipid regulation.

Liquid chromatography linked with tandem mass spectrometry (LC-MS/MS) has become a powerful technique for profiling lipidomes in complex samples⁹. Several strategies, coupled with chemoselective reactions, have been developed to facilitate the detection of lipids or differentiate lipid isomers¹⁰⁻¹². However, quantitative strategies for lipidomics remain limited. Currently, lipid quantification mainly relies on label-free methods¹³, which might suffer from analytical variations, long instrument time, and difficulty in preparing numerous isotope-incorporated standards. Alternatively, stable isotopic labeling, which introduces light and heavy isotopic reagents into analytes for relative quantification at the MS1 level¹⁴, is restricted to low-plexed analysis due to increased spectral complexity and limited availability of isotopic reagents.

Isobaric labeling is a powerful technique that enables quantitative analysis of multiple samples in one experiment¹⁵. This technique has been extensively used in proteomics and glycomics for high-throughput quantification using tandem mass tags (TMT)¹⁶, N,N-dimethyl leucine (DiLeu)¹⁷, or aminooxy tandem mass tags (aminoxyTMT)¹⁸, offering higher quantification accuracy, reproducibility, and sample throughput. Although several studies have attempted to utilize isobaric labeling for lipidomic quantification, typically via amine, carboxylate, or carbon-carbon double bonds^{19,20}, it has been difficult to target a wide range of PL classes in the complex biological milieu or develop a rapid and highly efficient approach that is crucial for sensitive, reproducible, and large-scale analysis of diverse lipid classes in complex biological samples.

Unlike peptides or glycans that have well-established bioconjugation via shared functional groups, the diversity of chemical structures of lipids presents challenges for targeting all PL classes. For functional groups shared among PL classes, such as phosphodiester and aliphatic groups, only limited reactions have been examined for direct conjugation for biological applications²¹. Diazo reagents have been reported to have broad and tunable reactivity to alkylate oxygen, nitrogen, and even carbon, which shows great potential to conjugate phosphodiesters. However, selective O-alkylation of phosphodiester for subsequent labeling is a difficult task due to their poor nucleophilicity and the presence of more reactive nucleophiles on PL structures²². Accordingly, it is desirable to develop compounds and linkers that allow for efficient labeling and subsequent analysis of a wide range of lipids.

### SUMMARY OF THE INVENTION

This invention provides linkers and methods for the functionalization and labeling of molecules, including but not limited to phosphate-containing and sulfate-containing molecules. In an embodiment, the linker comprises both a ketone functional group and a diazo functional group. Preferably the diazo group is able to react with the molecule (or an intermediary product formed by the molecule) thereby attaching the remaining portion of the linker to the molecule, where the ketone group remains available for further reactions or attachment to isotopic, isobaric, fluorescent, and/or chemical tags.

An aspect of the invention provides a linker, preferably a diazobutanone linker, capable of conjugating with phosphate groups (including phosphodiester groups) and sulfate groups on biomolecules using the diazo group, thereby enabling the functionalization of phosphate and sulfate-containing biomolecules. The linker exhibits high derivatization efficiency and chemoselectivity, accommodating a variety of other functional groups on the biomolecules. In an embodiment, the linker is made via a relatively simple two-step process and functionality enables direct oxygen-alkylation of lipid phosphate and lipid sulfate groups. The conjugated biomolecule is then reacted through the attached linker with tagging reagents via oxime bond formation as known in the art, including but not limited to isobaric and isotopically labeled tags, thereby generating a functionalized biomolecule containing a derivatized biomolecule, a linker portion, and tagging reagent, where one end of the linker portion is attached to the derivatized biomolecule and the second end of the linker is attached to the tagging reagent. Once labeled with the tagging reagent, traditional mass spectrometry and associated methods can be used for the analysis of the functionalized biomolecules. By coupling the linker with isobaric mass tags, multiplexed quantitative analysis can be achieved for an extensive variety of biomolecules and lipids.

In an embodiment, the present invention provides a method for functionalizing a biomolecule comprising a phosphate group or sulfate group, where the method comprises the steps of:
a) contacting the biomolecule with a linker comprising a first region having a diazo group and a second region having a ketone group, thereby reacting the first region of the linker with the phosphate group or sulfate group of the biomolecule and generating a conjugated biomolecule; and
b) contacting the conjugated biomolecule with a tagging reagent, thereby reacting the second region of the linker to the tagging reagent and generating a functionalized biomolecule labeled with the tagging reagent. In an embodiment, reacting the first region of the linker with the phosphate group or sulfate group of the biomolecule forms an alkylated phosphate or alkylated sulfated group.

Preferably, the linker has the following formula:

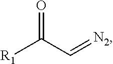

wherein R1 is hydrogen, or an alkyl group or aromatic ring having 12 carbon atoms or less, having 8 carbon atoms or less, or having 6 carbon atoms or less. In an embodiment, R1 is an alkyl group having 1 to 12 carbon atoms, 1 to 8 carbon atoms, 1 to 6 carbon atoms, or 1 to 3 carbon atoms. In an embodiment, R1 is an aromatic ring having 3 to 12 carbon atoms, 3 to 8 carbon atoms, 3 to 6 carbon atoms, or 3 to 5 carbon atoms.

In an embodiment, the linker has the following formula:

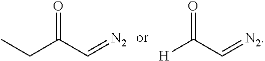

In an embodiment, reacting the first region of the linker with a phosphate group or sulfate group of the biomolecule further comprises removing excess amount of the linker by vacuum. This allows excess amounts of the linker to be removed without cumbersome separation steps that may also reduce the yield of the resulting conjugated biomolecule. Preferably, the linker is volatile at room temperature. In an embodiment, the linker has a boiling point less than 100° C., preferably a boiling point less than 75° C., more preferably a boiling point less than 65° C. (all temperatures at 1 atm).

In an embodiment, reacting the first region of the linker with the phosphate group or sulfate group of the biomolecule has a derivatization efficiency of 85% or greater, 90% or greater, or 93% or greater.

An aspect of the invention comprises generating the linker prior to contacting the linker with the target molecule. In an embodiment, generating the linker comprises the steps of functionalizing a dione to contain a diazo functional group and treating the functionalized dione with a basic solution, thereby generating a linker having a first region having the diazo group and a second region having a ketone group. Preferably, this step of generating the linker has a reaction yield of 60% or greater, 75% or greater, or 85% or greater.

In an embodiment, molecules able to be functionalized with the linker and subsequently labeled with a tagging reagent are molecules containing phosphate and/or sulfate groups. Preferably, the molecules are biomolecules including, but not limited to, phosphate-containing and/or sulfate-containing metabolites, lipids, carbohydrates, polypeptides, nucleic acids, and combinations thereof. Optionally, the molecules include, but not limited to, phosphate-containing lipids, phosphate-containing metabolites, sulfate-containing lipids, sulfate-containing metabolites, and combinations thereof.

Tagging reagents suitable for use with the present invention include any tagging reagent or isotopically labeled tag able to be attached to the linker after the linker has been conjugated to the biomolecule, for example, by reacting the second region of the linker having the ketone group. In an embodiment, the tagging reagent comprises an aminooxy group able to form a reaction with the ketone group of the linker via oxime bond formation. Preferably, the tagging reagent comprises at least one atom that is isotopically labeled. Suitable isotopically labeled tagging reagents include, but are not limited to, tandem mass tags (“TMT”), dimethylated amino acid tags (such as dimethylated leucine “DiLeu”), and aminooxy tandem mass tags (“aminoxyTMT”). Preferably, the tagging reagent is an aminoxyTMT since the aminooxy group can efficiently conjugate with the ketone group on the linker. Optionally, the tagging reagent comprises a fluorescent agent (i.e., a fluorophore) or a radioactive tag able to track the presence and position of a tagged biomolecule.

In an embodiment, generating the labelled biomolecule by reacting the second region of the linker to the tagging reagent has a labeling efficiency of 90% or greater, 95% or greater, or 99% or greater.

After labeling the biomolecule with the tagging reagent, the labeled biomolecule is able to be analyzed, such as by mass spectrometry analysis. The mass spectrometry analysis may include MS1 analysis as well as MS2 analysis where the labeled biomolecule is fragmented. Additional tagging reagents having the same mass can be used to label biomolecules in additional samples. The different samples are optionally combined, and the relative amounts of the labeled biomolecules compared. One of the samples may be a biomolecule present in known amount, allowing the relatively quantitative amounts of target biomolecules from the other samples to be determined

In an embodiment, the tagging reagent is an isobaric tag comprising a reporter group, a balance group, and a carbonyl-reactive group, where one or more atoms in the reporter group, balance group, or both, are isotopically heavy versions of the atom. In an embodiment, two or more isobaric tags are added to multiple samples containing the target biomolecules in order to compare and even quantify the amount of the target biomolecule in each sample. For example, in an embodiment the overall masses of the different tagging reagents are the same, but the mass of the different reporter groups will be different for each tagging reagent. As a result, the different samples can be analyzed using MS1 mass spectrometry and even combined. The exact mass of identical biomolecules from different samples presents a single peak at MS1 spectra but after fragmentation each biomolecule labeled with a different isobaric tag will generate a fragment having a reporter group with a different mass that is distinguishable from biomolecules labeled with a different tag from the different samples.

In an embodiment, the invention comprises two or more samples, wherein each sample contains an amount of the target biomolecule. The biomolecule in each of the two or more samples is contacted with the linker thereby generating a conjugated biomolecule in each of the two or more samples. The conjugated biomolecule in each of the two or more samples is then labeled with two or more tagging reagents (one tagging reagent for each sample), where each of the two or more samples is labeled with a different tagging reagent. Preferably, each of the different tagging reagents comprises a reporter group and a balance group, where one or more atoms in the reporter group, balance group, or both, are isotopically heavy versions of the atom. The reporter group of each of the different tagging reagents has a different mass due to differently isotopically labeled atoms in each reporter group, and the balance group of each of the different tagging reagents has a different mass due to the differently isotopically labeled atoms in each balance group. However, the overall total mass of the reporter groups plus the balancing group for each tagging reagent is the same. In a further embodiment, the labeled biomolecules in each of the two or more samples are fragmented and the resulting fragments are analyzed. Preferably, the reporter ion intensities of the labeled biomolecule are quantified in each of the two or more samples, such as through the use of a known standard.

A further embodiment comprises labeling target biomolecules within three or more samples with three or more tagging reagents; labeling target molecules within four or more samples with four or more tagging reagents; labeling target molecules within five or more samples with five or more tagging reagents; labeling target molecules within six or more samples with six or more tagging reagents; labeling target molecules within seven or more samples with seven or more tagging reagents; and labeling target molecules within eight or more samples with eight or more tagging reagents.

An aspect of the invention provides a kit is for multiplexed analysis of a target biomolecule. In an embodiment, the kit comprises a linker and two or more tagging reagents, where the linker has the following formula:

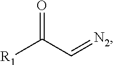

where each tagging reagent comprises a reporter group, a balance group, and a carbonyl-reactive group wherein one or more atoms in the reporter group, balance group, or both, are isotopically heavy versions of the atom. The reporter group of each tagging reagent has a mass different than the reporter groups of the other tagging reagents, the balance group of each tagging reagent has a mass different than the balance groups of other tagging reagents, and the overall mass of the reporter group plus the balance group for each tagging reagent is the same. Within the linker, R1 is hydrogen, or an alkyl group or aromatic ring having 12 carbon atoms or less, having 8 carbon atoms or less, or having 6 carbon atoms or less. In an embodiment, R1 is an alkyl group having 1 to 12 carbon atoms, 1 to 8 carbon atoms, 1 to 6 carbon atoms, or 1 to 3 carbon atoms. In an embodiment, R1 is an aromatic ring having 3 to 12 carbon atoms, 3 to 8 carbon atoms, 3 to 6 carbon atoms, or 3 to 5 carbon atoms.

Preferably, each tagging reagent comprises an aminooxy group able to form a reaction with a ketone group in a conjugated biomolecule to be labeled. In an embodiment, the linker has the following formula:

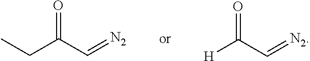

An aspect of the invention provides a functionalized compound or labeled biomolecule, including but not limited to functionalized lipids and metabolites. In an embodiment, the invention provides a compound comprising the formula:

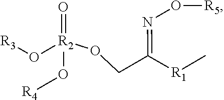

where,
R1 is an alkyl group or an aromatic ring having 12 carbon atoms or less, preferably 8 carbons, or less or 6 carbons or less;
R2 is P or S;
R3 and R4 are, individually from each other, hydrogen, a linear or branched hydrocarbon, a lipid, an aliphatic group, or comprises polar functional group; and
R5 is a tagging reagent.
   In an embodiment, R1 is —CH₂—.

Preferably, R3 is an aliphatic region or part, preferably having the formula:

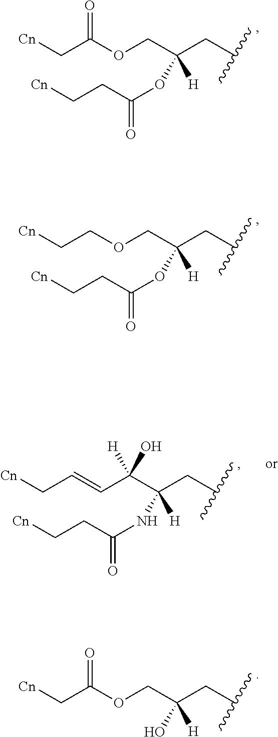

Preferably, R4 is a head group having the formula:

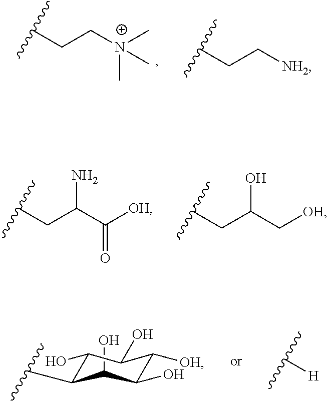

wherein when R2 is S R4═H.

In an embodiment, a target biological molecule in two or more samples is labeled and subsequently analyzed using the tagging reagents of the present invention, where at least one sample is a biological sample taken from a patient before a treatment is administered to the patient, and one or more samples are biological samples taken from the patient at one or more time periods after the treatment has been administered to the patient. The sample taken from the patient may include, but is not limited to, a fluid sample (such as blood), cell sample, or tissue sample (e.g., tissue biopsy). In an embodiment, the treatment is the administration of a drug or therapeutic which may result in the increase or decrease of a biological molecule or metabolite.

Currently, there are no commercial tag reagents available that can enable isobaric labeling for multiplexed quantitative lipidomics due to the extensive diversity in lipid chemical structures. Previous approaches have utilized commercial Tandem Mass Tag (TMT) reagents or aziridination for isobaric labeling in lipidomic analysis. However, these techniques either limit the analysis to a restricted selection of lipid classes or exhibit low efficiency. In contrast, the present invention employs diazo-based linkers, which are able to target all lipids containing phosphate and sulfate groups, encompassing more than ten lipid classes. This approach offers a robust and multiplexed quantitative analysis method for lipidomics. The labeling procedure can be completed within a span of two hours and involves simple cleanup steps, thereby reducing sample loss and simplifying experimental operations. The rapid procedure and cost-effective materials support large-scale, high-throughput analysis, providing notable advantages for extensive lipidomic studies.

### BRIEF DESCRIPTION OF THE DRAWINGS

FIG. 1 shows a two-step derivatization strategy using an in-house synthesized carbonyl-containing diazo linker, diazobutanone, in an embodiment of the present invention. The phosphate group of a lipid is derivatized with diazobutanone to introduce a tag-reactive group to the lipid. An aminoxyTMT tag is coupled to the tag-reactive group during an isobaric labeling step, and the resulting products were subjected to LC-MS/MS analysis.

FIG. 2 shows MALDI mass spectra of phospholipids directly extracted from cells (top), phospholipids (PL) after derivatization where 70 Da mass increments were introduced (middle), and PL derivatives labeled with AminoxyTMT leading to 296 mass increments (bottom).

FIG. 3 shows (panel A) six classes of identified phospholipids in label-free samples and in samples labeled using diazobutanone and the aminoxyTMT tags. Panel B shows quantitative results with a mixing ratio 1:2:4:6:8, and panel C shows fragment characterization of PG 16:0_18:1 using HCD.

FIG. 4 shows a diazobutanone-assisted isobaric labeling scheme, structures of lipid classes, and AminoxyTMT in an embodiment of the present invention. Prior to labeling, phospholipids and sulfated glycolipids generally form negatively charged ions. However, following phosphate derivatization and AminoxyTMT labeling which contains a tertiary amine group, the labeled lipids can be readily analyzed in the positive ion mode.

FIG. 5 shows a synthesis scheme of diazobutanone in an embodiment of the invention. Detailed synthetic methods are described in Example 7.

FIG. 6 shows full MS scan spectra of derivatization of PL mixtures by new and 60-day-old diazobutanone. The peaks on the left are the internal standards. Nearly no differences were shown between the two spectra, indicating good stability of diazobutanone.

FIG. 7 shows derivatization condition optimized by reaction solvents (panel a), concentrations of HBF₄ (panel b), concentration of diazobutanone (panel c), and reaction time using a lipid mixture consisting of PE 18:1_18:1, PC 16:0_18:1, PS 18:1_18:1, PG 18:1_18:1, PI 18:1_18:1, and PA 18:1_18:1 (panel d).

FIG. 8 shows an evaluation of the diazobutanone-assisted isobaric labeling from FIG. 4. Panel a) shows information on diagnostic ions, reaction efficiency and sensitivity for the labeled PLs from 10 lipid classes. The detailed LOQ information and the lipid standard list are provided in Tables 1 and 2. Panel b) shows linear correlation between the measured ratios of reporter ion intensities and the amount ratios of lipid standards. Panel c) shows extracted LC chromatography of the labeled PL, PG 18:1_18:1, with 6 different channels from the lipid mixture with the ratio 1:1:2:4:6:8, showing no retention time shift among each other.

FIG. 9 shows a diazobutanone-assisted isobaric labeling of lipid standards and MS2 fragmentation of labeled lipids in an embodiment of the invention. Panel a) shows spectra of unreacted lipids in the negative ion mode (top panel), derivatized lipids by diazobutanone in the positive ion mode (middle panel), and AminoxyTMT-labeled lipids (bottom panel). Panel b) shows MS2 spectra of representative labeled lipids, PE 15:0_18:1(d7) and PG 16:0_18:1.

FIG. 10 shows MS2 spectra of all lipid standards from 10 lipid classes by HCD (NCE 32%). Diagnostic ions for class identification are highlighted. Lipid standards included PC 15:0-18:1(d7), ether PC 16:0-18:1, SM d18:1-18:1(d9), PE 15:0-18:1(d7), Lyso PC 18:1(d7), PA 15:0-18:1(d7), PG 15:0-18:1(d7), PS 15:0-18:1(d7), PI 15:0-18:1(d7), and 3-O-Sulfo MGDG 18:1 representing SGL

FIG. 11 shows MS2 spectra of labeled PE 15:0_18:1(d7) and PG 16:0_18:1 and the proposed structures for the fragment ions. Diagnostic ions are unique to the lipid classes. Acylium ions and acylium glycerol ions indicate the fatty acid chains of the lipids.

FIG. 12 shows MS3 spectrum of PA 18:1_18:1. MS3 analysis was performed on an Orbitrap Elite mass spectrometer. The highlighted peak represents FA 18:1 acylium ion, indicating the acyl chain information of the precursor ion.

FIG. 13 shows MS2 spectrum of PG 40:7. The highlighted peaks are diagnostic ions for class identification or acyl chain elucidation. PG species tend to generate [PG+H—RCOO]+ and show less efficiency on PG diagnostic ion, [PGhead+Tag+H]+.

FIG. 14 shows MALDI MS spectra of unreacted PL extract (top panel), derivatized PL extract (middle panel), and labeled PL extract (bottom panel). Representative peaks are highlighted and marked. Due to the high ionization efficiency of PC species on MALDI, PCs are indicated.

FIG. 15 shows a diazobutanone-assisted two-step isobaric labeling and mass spectrometry workflow in an embodiment. Deuterated lipids were added prior to sample homogenization, which will be used for lipid amount estimation and the correction of lipid extraction recovery. Six samples will be labeled separately and pooled prior to MS analysis.

FIG. 16 shows performance of diazobutanone-assisted isobaric labeling on complex samples in an embodiment. Panel a) shows identified PL species from 6 central PL classes in mouse liver samples. Panel b) shows a Venn diagram of identified PL species from biological triplicates. Panel c) shows reporter ions of the deuterated PL standards with a ratio of 1:2:4:4:2:1 in the mixture of the biological samples. Panel d) shows reporter ion ratios for 1:1:1:1:1:1 mixture. Box plots demarcate the median (line), the 25th and 75th percentile (box), and the 5th and 95th percentile (whiskers). Panel e) shows the box plots of the labeled mixture with a ratio of 1:1:2:4:6:8 and 6 label-free (LF) with ratios of 1:1:2:4:6:8.

FIG. 17 shows MS2 spectra of labeled PI 36:2. In the spectrum on the top, only FA 18:1 acylium ion was detected, and thus the reporter ratio was used for PI 18:1_18:1 quantification. In the middle panel, acylium ions indicating two PI 36:2 acyl chain compositions, and the quantification was marked as PI 18:1_18:1/18:0_18:2. In the bottom spectra, only FA 18:0 and FA 18:2 acylium ions were detected, the reporter ions in the spectrum were used for PI 18:0_18:2.

FIG. 18 shows the quantitative lipidomics of lean and obese mouse liver tissues. Panel a) shows the identified PL species from liver samples. Panel b) shows PC/PE ratios in 3 lean and 3 obese mice. Panel c) shows hierarchical cluster analysis discriminated healthy and obese mice by quantitative analysis of PLs. Panel d) shows tandem MS spectra of representative PLs with shorter acyl chains (PI 16:0_18:2; upper panel) and longer acyl chains (PS 18:0_22:6; lower panel).

### DETAILED DESCRIPTION OF THE INVENTION

### Definitions

As used herein the terms “tagging” and “labeling” refers to reacting a reagent or compound with a target molecule of interest, including but not limited to lipids and metabolites containing a phosphate group or sulfate group, so that one or more functional groups are attached to the molecule of interest. A “tagged” or “labeled” target molecule refers to a molecule of interest having the one or more functional groups attached.

As used herein, “lipids” refer to hydrophobic and amphiphilic organic molecules, including fatty acids, sterols, fat-soluble vitamins, monoglycerides, diglycerides, triglycerides, sphingolipids, phospholipids, and sulfolipids.

As used herein, “metabolites” refer to small molecules that are an intermediate or end product of a biological process, such as the breakdown or chemical modification of a precursor compound, and that have a molecular weight of less than 1,000 daltons.

A “ketone” generally refers to an organic compound having a functional group with the structure RC(═O)R′, where R and R′ can be a variety of carbon-containing substituents.

A “diazo group” generally refers to an organic compound having formula:

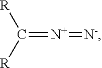

wherein one R group is hydrogen and the other R group is an alkyl group.

The term “alkyl” refers to a monoradical of a branched or unbranched (straight-chain or linear) saturated hydrocarbon and to cycloalkyl groups having one or more rings. Alkyl groups as used herein include those having from 1 to 12 carbon atoms, preferably having from 1 to 6 carbon atoms. Alkyl groups include small alkyl groups having 1 to 3 carbon atoms. Cycoalkyl groups include those having one or more rings. Cyclic alkyl groups include those having a 3-, 4-, 5-, 6-, 7-, 8-, 9-, 10-, 11- or 12-member carbon ring and particularly those having a 3-, 4-, 5-, 6-, or 7-member ring. The carbon rings in cyclic alkyl groups can also carry alkyl groups. Alkyl groups are optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms.

An “alkoxy group” is an alkyl group linked to oxygen and can be represented by the formula R—O. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups.

The term “aromatic” refers to a hydrocarbon having a conjugated cyclic molecular structure. Aryl groups include those having from 3 to 12 carbon atoms, 3 to 8 carbon atoms, 3 to 6 carbon atoms, or 3 to 5 carbon atoms. Aryl groups can contain a single ring (e.g., phenyl), one or more rings (e.g., biphenyl) or multiple condensed (fused) rings, wherein at least one ring is aromatic (e.g., naphthyl, fluorenyl, or anthryl). Heterocyclic aromatic rings can include one or more N, O, or S atoms in the ring, and can include those with one, two or three N, those with one or two 0, and those with one or two S, or combinations of one or two or three N, O or S. Aryl groups are optionally substituted. Substituted aryl groups include among others those which are substituted with alkyl or alkenyl groups, which groups in turn can be optionally substituted.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

As used herein, “isotopically labeled”, “isotopically enriched”, “isotopic composition”, “isotopic”, “isotope”, and the like refer to compounds (e.g., tagging reagents, labeled molecules, labeled samples, and end-products, etc.) whereby a process has introduced one or more isotopes into the relevant compound in excess of the natural isotopic abundance. “Isotopically-heavy” refers to a compound or fragments/moieties thereof that have been enriched with one or more high mass, or heavy isotopes (e.g., stable isotopes such as deuterium, ¹³C, ¹⁵N, and ¹⁸O).

In an embodiment, an isotopically labeled molecule or sample comprises a specific isotopic combination (i.e., isotopically heavy versions of one or more atoms) present in an abundance that is at least 10 times greater, for some embodiments at least 100 times greater, for some embodiments at least 1,000 times greater, for some embodiments at least 10,000 times greater, than the abundance of the same compound having the same isotopic combination in a naturally occurring or unenriched sample. In another embodiment, an isotopically enriched sample has a purity with respect to a compound of the invention having a specific isotopic composition that is substantially enriched, for example, a purity equal to or greater than 90%, in some embodiments equal to or greater than 95%, in some embodiments equal to or greater than 99%, in some embodiments equal to or greater than 99.9%, in some embodiments equal to or greater than 99.99%, and in some embodiments equal to or greater than 99.999%. In another embodiment, an isotopically enriched sample is a sample that has been purified with respect to a compound of the invention having a specific isotopic composition, for example using isotope purification methods known in the art.

“Fragment” refers to a portion of molecule, such as labeled phospholipid or sulfolipid. Fragments may be singly or multiply charged ions. Fragments may be derived from bond cleavage in a parent molecule, including site specific cleavage of polypeptide bonds in a parent molecule. Fragments may also be generated from multiple cleavage events or steps. Fragments useful in the present invention include fragments formed under metastable conditions or result from the introduction of energy to the precursor by a variety of methods including, but not limited to, collision induced dissociation (CID), higher-energy collision dissociation (HCD), surface induced dissociation (SID), laser induced dissociation (LID), electron capture dissociation (ECD), electron transfer dissociation (ETD), ultraviolet photo-dissociation (UVPD), or any combination of these methods or any equivalents known in the art of tandem mass spectrometry. Fragments useful in the present invention also include, but are not limited to, x-type fragments, y-type fragments, z-type fragments, a-type fragments, b-type fragments, c-type fragments, internal ion (or internal cleavage ions), immonium ions or satellite ions. The types of fragments derived from a parent analyte often depend on the sequence of the parent, method of fragmentation, charge state of the parent precursor ion, amount of energy introduced to the parent precursor ion and method of delivering energy into the parent precursor ion. Properties of fragments, such as molecular mass, may be characterized by analysis of a fragmentation mass spectrum.

### Overview

MS-based quantitative lipidomics is an emerging field aiming to uncover the intricate relationships between lipidomes and disease development. However, quantifying lipidomes comprehensively in high-throughput manners remains challenging due to the diverse lipid structures. This invention provides diazo linkers and compounds, including but not limited to diazobutanone, that enables a diazo compound-assisted isobaric labeling strategy for multiplexed quantification across a broad range of molecules, including various phospholipids, glycolipids, and small molecules, such as metabolites.

The diazo linkers and compounds are designed to conjugate with phosphate, phosphodiester, and sulfate groups, while accommodating various functional groups on different molecules and lipid classes, enabling subsequent isobaric labeling for high-throughput multiplex quantitation. This diazo compound-assisted method demonstrates excellent performance in terms of labeling efficiency, detection sensitivity, quantitative accuracy, and broad applicability to various biological samples.

For example, the diazo linkers described herein enable direct conjugation to both phosphodiester and sulfate groups under the mild conditions. The ketone group on the linker can conjugate with aminooxy groups specifically and efficiently to facilitate the functionalization of these biomolecules. Traditionally, existing approaches initiate with phosphine or phosphoramidite to synthesize phosphotriester groups, instead of directly O-alkylation of phosphate groups. This not only necessitates numerous synthetic steps but also constrains the applicability to biological samples.

In particular, the diazobutanone linker described herein has both a diazo group and a ketone group. By coupling the diazobutanone linker with isobaric mass tags, multiplexed quantitative analysis can be achieved for an extensive variety of lipid classes. The performance of this diazobutanone-assisted isobaric labeling method has been benchmarked, demonstrating exemplary performance in terms of labeling efficiency, detection sensitivity, quantitative accuracy, and broad applicability to various biological samples. The labeling of lipids can generate reporter ions for multiplexed quantification, diagnostic ions for class identification, and acylium ions for fatty acid chain elucidation. This empowers a high lipidome coverage and comprehensive quantitative lipidomics. Notably, this method represents the first study to achieve multiplexed quantification of a wide range of lipid classes simultaneously on a global scale from multiple complex biological samples.

The diazobutanone linker can be synthesized conveniently in two steps, resulting in a high-yield and high-purity product. This linker is economically efficient owing to high product yields and the use of cost-effective, commercially available reagents. The linker was designed to be compact and volatile, which facilitates easy cleanup using a vacuum and is compatible with downstream mass spectrometry analyses. Furthermore, this linker displays high stability that is compatible with biomolecules and suitable for use under standard experimental conditions. The synthesized diazobutanone can be stored at −20° C. over extended periods without reducing its reactivity.

The derivatization efficiency of the diazobutanone linker has been observed to exceed 97% for phosphodiester groups and 93% for sulfate groups within a 30-minute reaction. In addition, the linker exhibits a high chemoselectivity towards phosphate and sulfate groups under optimized conditions, which accommodates various functional groups on biomolecules such as ester, ether, hydroxyl, amine, amide, carboxylate, carbon-carbon double bond, and quaternary amine groups. Such compatibility maximizes the yield of the desired product and minimizes side reactions that could potentially complicate analysis and decrease the sensitivity towards target lipids.

### Example 1—Cell Extract Labeling

Phospholipids are essential biomolecules that are involved in many biological processes. These functions are determined by their chemical structures and the relative abundance of each species. The disturbance of phospholipids is associated with many diseases. Quantification of phospholipids will provide a deeper insight into their functions and roles in disease mechanisms.

Isobaric labeling strategy enables high-throughput quantification without increasing spectral complexity. Quantification is achieved on MS2 level by generating unique masses from the isobaric tags. This strategy has been widely used in quantitative proteomics and glycomics. Despite the attempt to utilize this strategy in lipidomics, the studies have been done only to limited classes of lipids due to the high structural diversity of lipids.

The goal of this example was to develop a derivatization strategy to enable the labeling with isobaric mass tags to achieve high-throughput quantification for all major phospholipid classes.

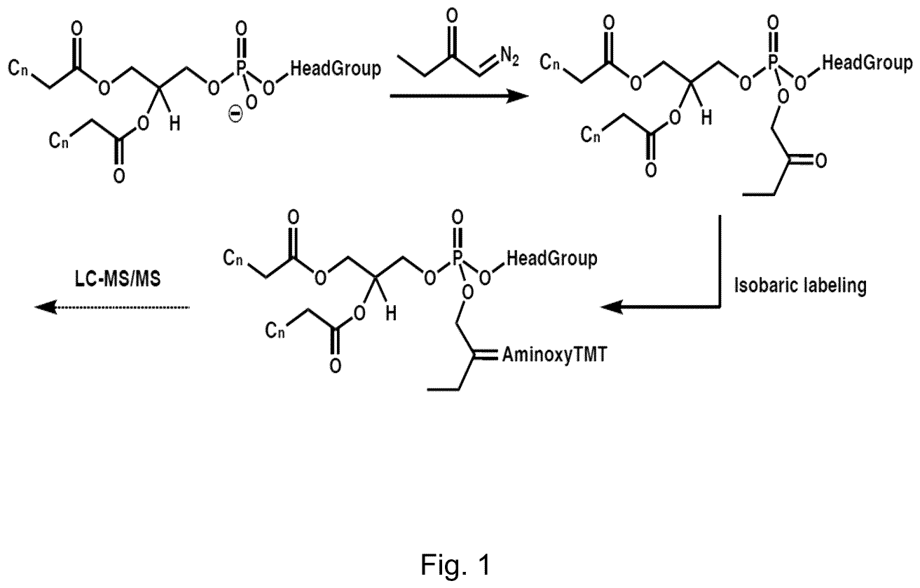

A two-step derivatization strategy was developed using an in-house synthesized carbonyl-containing diazo compound (diazobutanone) (FIG. 1). The diazo group can mainly react with phosphate groups under a specific condition. Phosphate groups were derivatized with diazobutanone to introduce a tag-reactive group for isobaric labeling. With careful optimizations, the derivatization and labeling efficiency were able to reach more than 95% for each phospholipid class with minimum side reactions. The resulting products were subjected to LC-MS/MS analysis to generate reporter ions for relative quantification and diagnostic ions to validate the phospholipid classes and aliphatic chains.

Cell lipids were extracted using Folch extraction. Label-free lipidomics was conducted on Elite Orbitrap in both positive and negative ion modes. The LC-MS data was processed using MS-Dial. To eliminate false identification, identified lipids are filtered by MS2 score and retention time. The identification results were used as a new database for labeled samples. For labeled lipidomics, the analysis was carried out in the positive ion mode. Besides exact mass matching with the label-free database, diagnostic ions in MS2 spectra were also considered to identify labeled lipids in cell extract. Reporter ions were used for relative quantification.

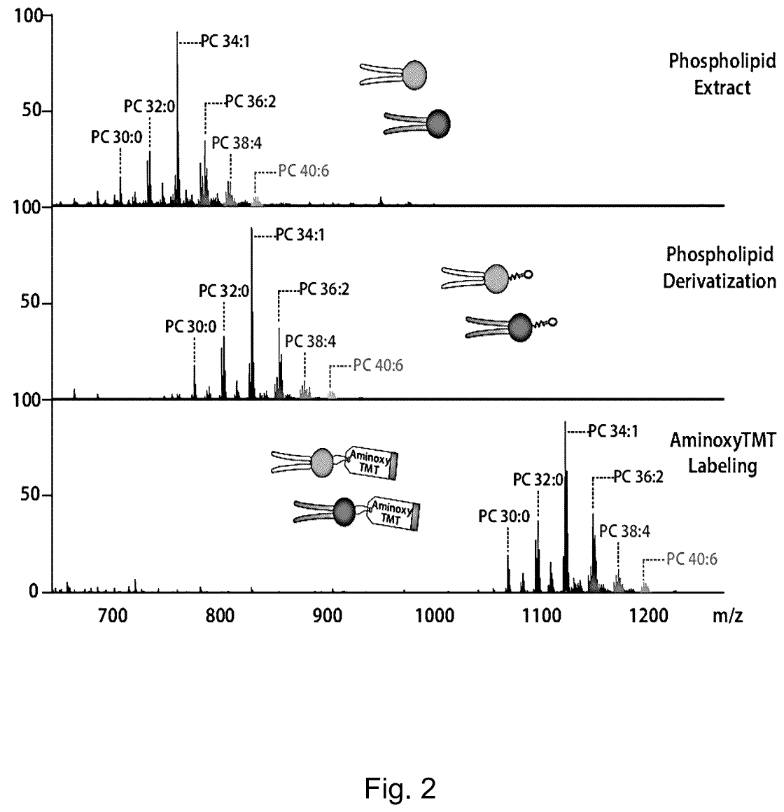

FIG. 2 shows MALDI MS spectra of phospholipids directly extracted from cells (top), phospholipids (PL) after derivatization where 70 Da mass increments were introduced (middle). Lastly, PL derivatives were labeled with AminoxyTMT leading to 296 mass increments (bottom). The reaction efficiencies were high for each step by examining the intensity of produces before and after reactions. Representative PCs were indicated in the spectra to show the mass shift in each step.

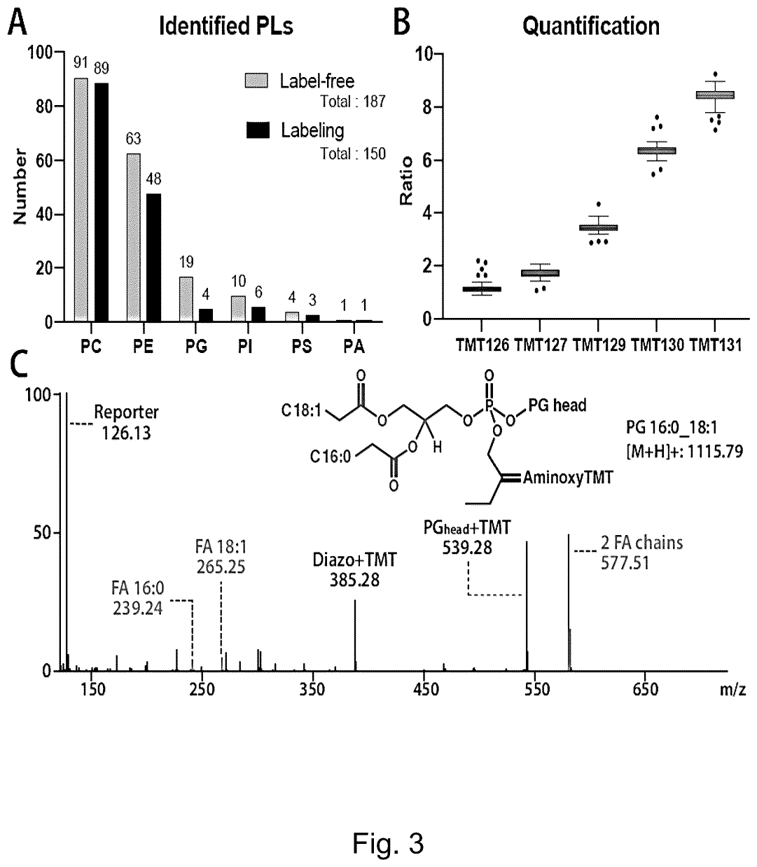

Six classes of phospholipids were successfully derivatized with a carbonyl-containing compound enabling the labeling by AminoxyTMT tags for multiplexed analysis (FIG. 3). This method features high labeling efficiency, quantitative accuracy, multiplexed quantification, and, most importantly, simultaneous analysis of all major phospholipid classes for high-throughput phospholipid quantification.

### Example 2—Design of Diazobutanone and AminoxyTMT Labeling

Diazobutanone, which can be synthesized in two steps and easily removed by vacuum after reactions, enables O-alkylation of phospholipids (PL) and specific reactions with aminooxy isobaric reagents. With optimized conditions, all lipids in a sample can be isobarically tagged under mild conditions, and the final products can generate reporter ions for relative quantification, diagnostic ions indicating lipid classes, and acylium ions for differentiating acyl chains.

Using aminoxyTMT, which is currently available up to 6-plex, six major classes of phospholipids were successfully profiled and quantified in liver tissues from three healthy lean mice and three insulin-resistant obese mice in a high-throughput fashion. Overall, 243 phospholipids were quantified in liver tissues, offering a universal and highly efficient method to quantify broad classes of phospholipids in complex biological samples.

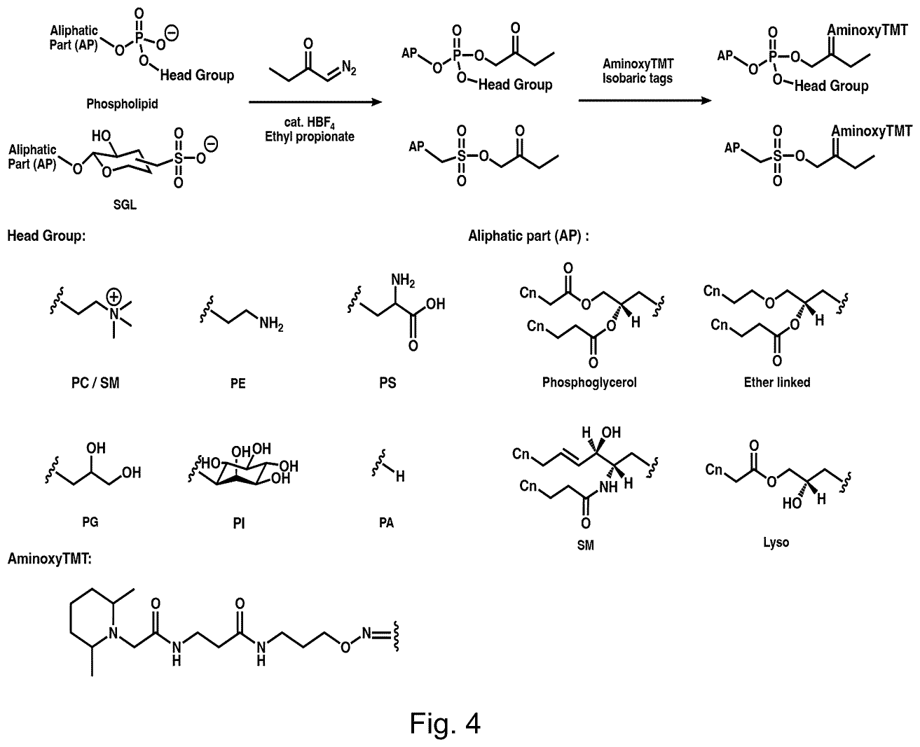

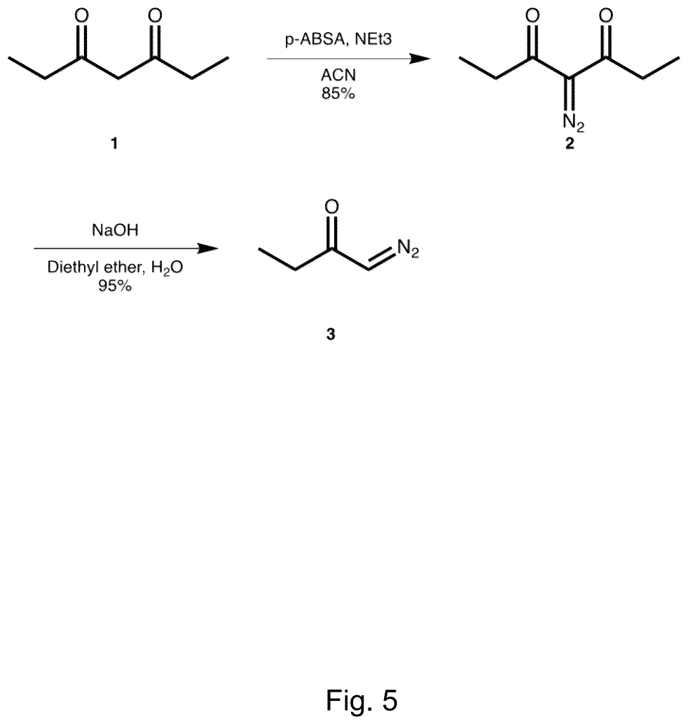

To develop a simple and applicable high-throughput method, a diazobutanone compound was designed that incorporates a diazo group for phosphodiester O-alkylation and a ketone group for subsequent isobaric labeling (see FIGS. 4 and 5). Oxime bond formation was selected for the labeling step in this study because oxime bond formation between carbonyl groups and aminooxy groups is highly specific and efficient, leaving other functional groups on phospholipids intact. Additionally, it was observed that the final products, phosphotriesters, are prone to hydrolysis under alkaline or reductive conditions while tolerating mild acidic conditions, where the oxime formation occurs²³.

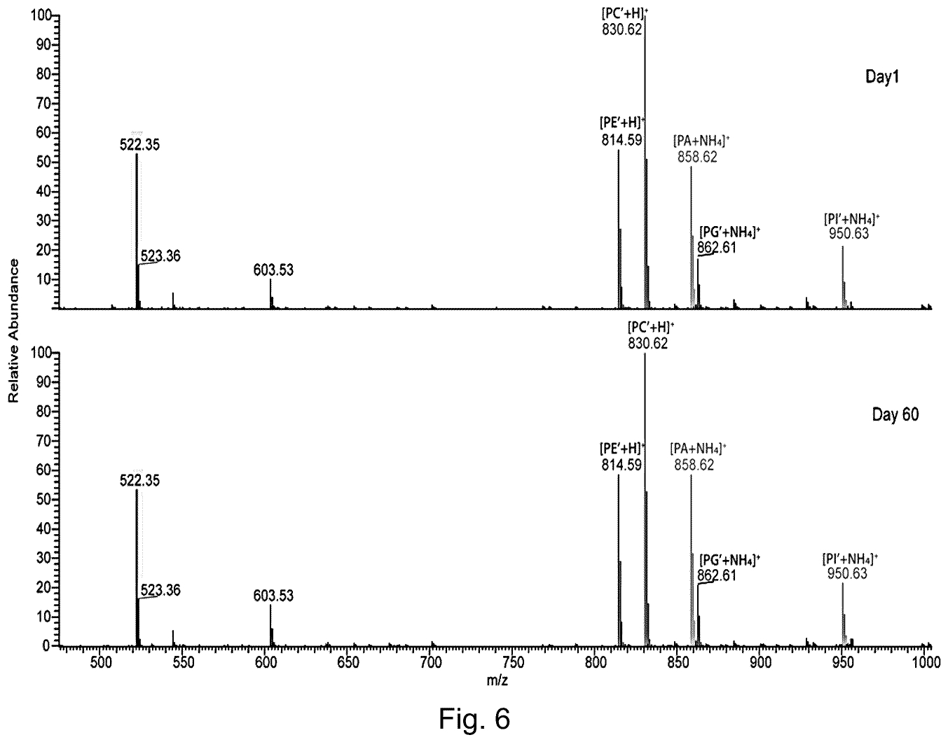

Critical to the properties of diazobutanone with biomolecules, the compound was designed to be volatile and placed carbonyl groups next to the diazo groups. Diazoalkanes, such as diazomethane, are typically toxic and explosively reactive, while diazo groups can be stabilized by delocalizing the electrons on the a carbon²⁴. Therefore, the use of carbonyl groups to diminish the reactivity of diazo groups makes the compound stable and compatible with biomolecules. Diazobutanone can be stored at −20° C. for months (60 days) without a decrease in reaction efficiency (FIG. 6). The volatility of diazobutanone allows for the removal of an excess amount of reagent by vacuum, simplifying the cleanup steps and reducing sample loss, which makes it compatible with subsequent mass spectrometry-based analyses.

To demonstrate the wide applicability of diazobutanone to various lipid classes, it was tested using representative lipid standards from nine phosphate-containing lipid classes, including phosphatidylcholine (PC), phosphatidylethanolamine (PE), phosphatidylglycerol (PG), phosphatidylserine (PS), phosphatidic acid (PA), phosphatidylinositol (PI), lysophosphatidylcholine (LysoPC), ether-phosphatidylcholines (etherPC), and sphingomyelin (SM). The lipid structures for each class are shown in FIG. 4. Besides phospholipid (PL) classes, it was also found that sulfate groups might hold similar reactivity to phosphate groups. Sulfated glycolipids (SGL) are enriched in the nervous and immune systems and exert unique functions from their non-modified glycolipids due to their negative charges. Thus, SGL was included in the method development²⁵.

In the initial tests, it was found that fluoroboric acid (HBF4) was the most efficient catalyst in the reactions, probably due to the generation of a non-coordinating counterion during the reaction²⁶. However, higher concentrations of HBF4 caused severe side reactions on multiple functional groups on lipids, such as the alkylation of amine, amide, or hydroxyl groups, while lower amounts of HBF4 failed to reach enough reaction yields. To promote efficiency without generating unwanted reactions, several condition factors were examined, such as temperature and solvent. The solvent effect was found to be crucial for the reactions. The diazobutanone reacted with the hydroxyl groups of PG in chloroform or showed low reactivity under ether systems. After testing all common reaction solvents, ethyl propionate, which is not commonly used for diazo compounds, generated minimum side reactions and obtained the highest signal intensity of lipid derivatives.

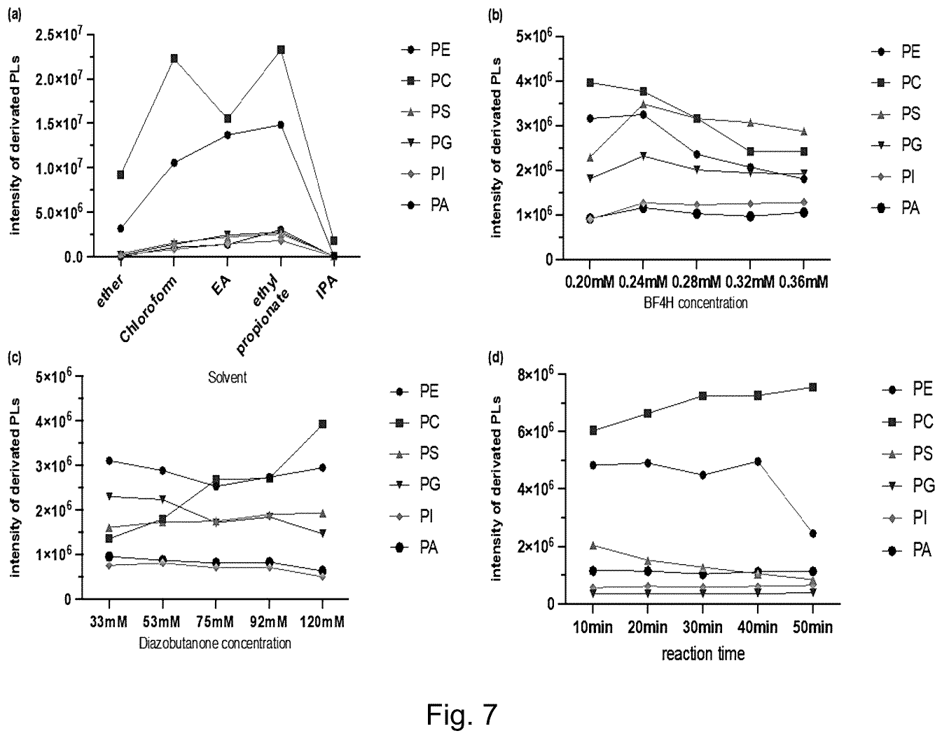

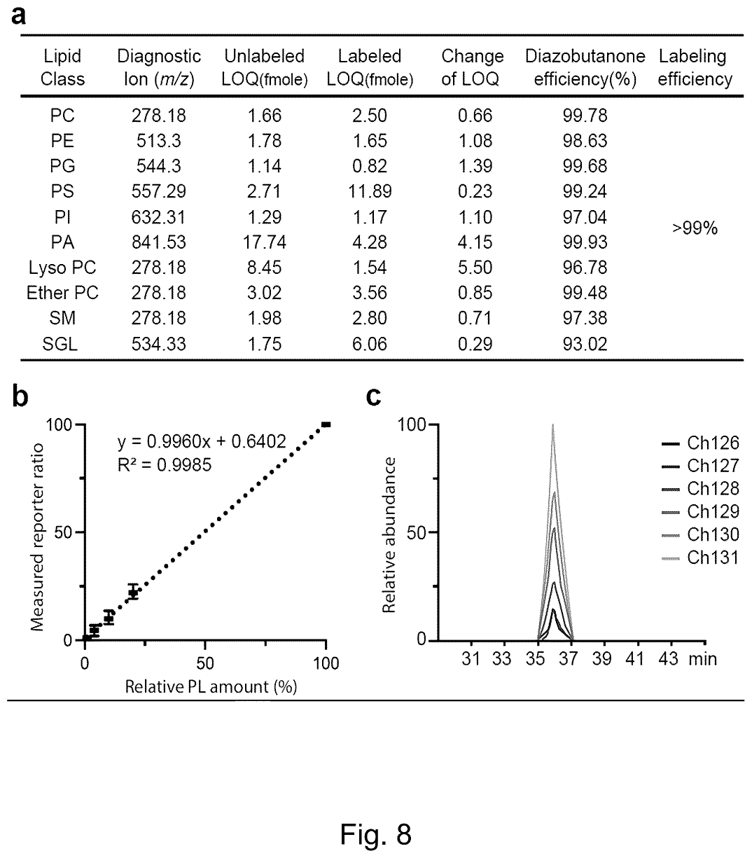

With careful optimizations (FIG. 7), a mild condition of 75 mM diazobutanone and 0.24 mM fluoroboric acid was used in ethyl propionate at room temperature for 35 min for subsequent experiments. An average of 98% conversion yields was achieved for all lipid standards with minimum side products (FIG. 8, panel a). The optimized reaction conditions exhibited high reaction efficiency and easy clean-up by vacuum, resulting in maximum conversions of lipid derivatives for quantitative analysis. It is worth noting that the selectivity of diazobutanone toward phosphate and sulfate groups under the optimized condition leaves other functional groups on lipids intact, such as ester, hydroxyl, amine, amide, carboxylate, carbon-carbon double bond, and quaternary amine groups, thus avoiding multiple side products resulting from different reactive sites that might complicate the analysis and lower the sensitivity of target lipids.

In the second step of the labeling, aminoxyTMT isobaric mass tags that are developed for targeting carbonyl-containing biomolecules were used to conduct isobaric labeling. Aminooxy groups of the tag reagents could specifically react with the ketone groups on the lipid derivatives that were introduced by diazobutanone. A lipid-to-aminoxyTMT ratio of 1:2 and reaction solvent of 10% isopropanol/methanol with 0.1% acetic acid were used. Near-complete labeling efficiencies was achieved for all lipid standards after reacting for 15 minutes and hydrolysis of PL products was not observed. The excess tag reagents can be removed by extraction using water and ethyl acetate. The whole labeling procedure could be completed within 2 hours, enabling fast and cost-effective experiments for high throughput quantification.

### Example 3—Characterization of Labeled Lipids

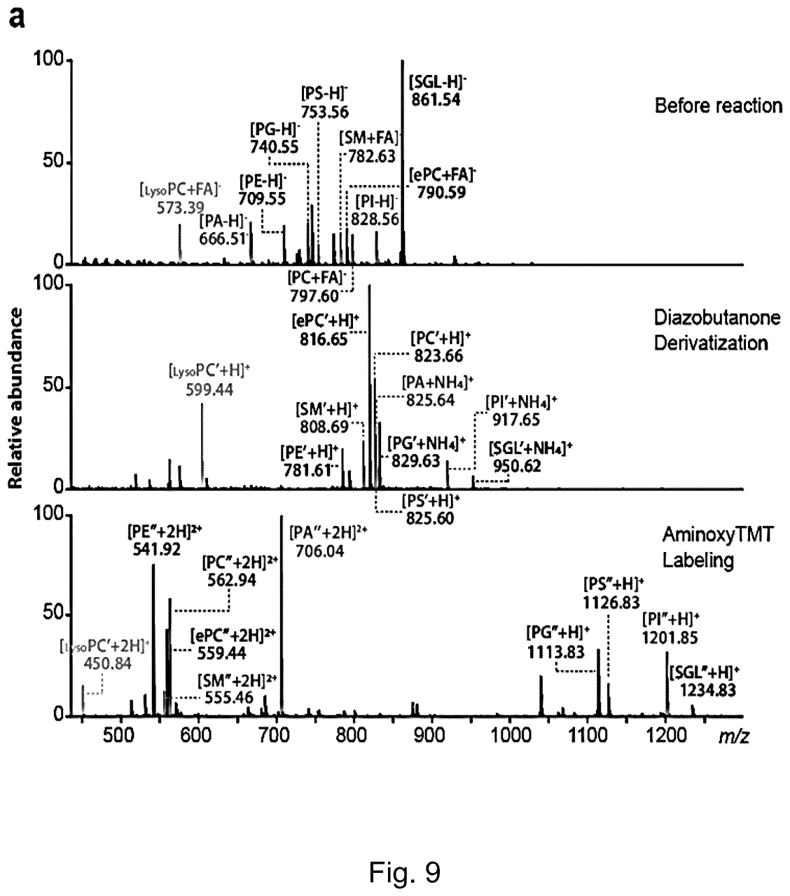

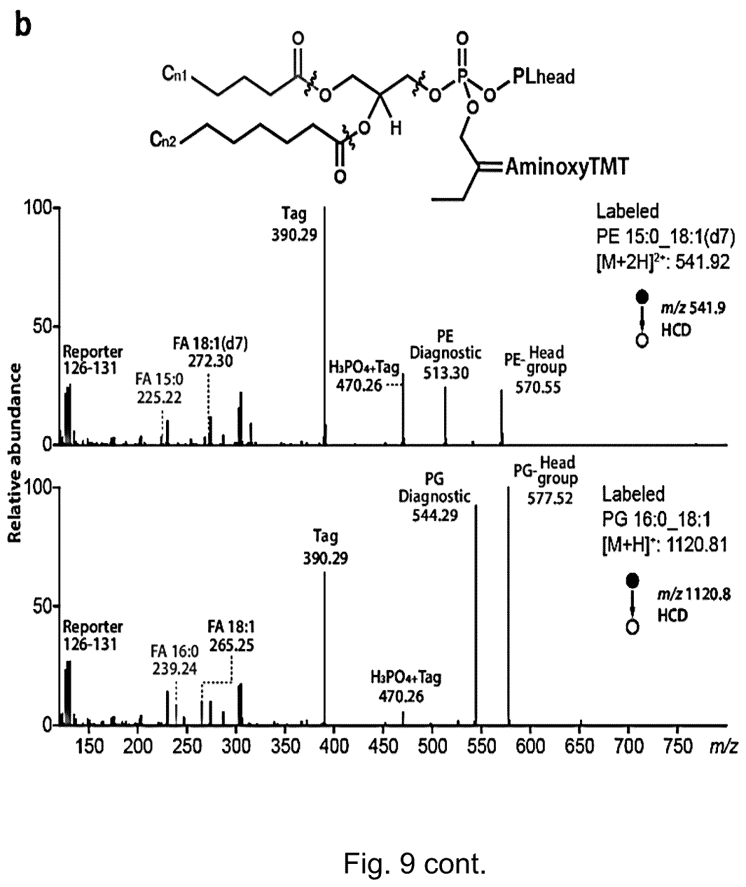

After labeling (FIG. 9, panel a), 372 Da mass increments were introduced to the lipid standards, which comprised of butanone plus aminoxyTMT. PA had a 744 Da mass increment, resulting from the incorporation of 2 units of tags, while some single-labeled PA product was also observed in low yields. In the previous reports involving diazomethane, alkylated amino and fragmented PL, particularly PS, products were generated, whose molecular weights are the same as the corresponding PC and PA^{26,27}. Since the products could potentially confound the detection of PC or PA, the separation of PLs based on PL classes were usually performed using hybrid solid-phase extraction columns. In this study, products with alkylation on amine groups were not observed, and PL fragmentation was observed to a lesser extent. A possible explanation for this could be the use of the lower-reactivity diazobutanone and milder reaction conditions compared to previous works. However, to avoid inaccurate quantitation of PA, it is desirable to separate PA from other PL classes during the analysis.

To assess the performance of this method, five different amounts of PLs (300 ng, 60 ng, 30 ng, 12 ng, and 3 ng) were labeled and analyzed. The correlation between the measured ratios of reporter ions and the expected ratios of lipids was examined (FIG. 8, panel b), revealing a linear relationship with an R² value of 0.9985 and a slope of 0.9960. The retention time of PLs labeled with different channels displayed no significant shift between each channel, ensuring simultaneous MS2 analysis of PLs with various tag variants (FIG. 8, panel c). The sensitivity of the method was also evaluated by comparing the limits of quantification (LOQs) before and after labeling using lipid standards (FIG. 8 and Table 1). The results indicated that the technique yielded LOQs comparable to those of unlabeled lipids, suggesting that the method successfully facilitates multiplexed quantification without compromising sensitivity.

TABLE 1
Information on reaction efficiency and sensitivity for the labeled
PLs from 10 lipid classes. Given that high-resolution Orbitrap MS2
data do not produce a blank/background response, we established the
limit of quantification (LOQ) following guidelines from the previous
literature. The signal responses were noted to be ten times higher
than the blank values. For the labeled phospholipids, apart from
examining signal responses, we also scrutinized MS2 spectra, where
we observed quality reporter ions and diagnostic ions.

| | Unlabeled | | Labeled | | | |
|---|---|---|---|---|---|---|
| Lipid | LOQ | | LOQ | | Diazobutanone | Labeling |
| Class | (fmole) | RSD | (fmole) | RSD | efficiency (%)^{a} | efficiency^{b} |
| PC | 1.66 | 0.12 | 2.50 | 0.38 | 99.78 | |
| PE | 1.78 | 0.16 | 1.65 | 0.07 | 98.63 | >99% |
| PG | 1.14 | 0.11 | 0.82 | 0.16 | 99.68 | |
| PS | 2.71 | 0.21 | 11.89 | 0.09 | 99.24^{c} | |
| PI | 1.29 | 0.21 | 1.17 | 0.09 | 97.04 | |
| PA | 17.74 | 0.06 | 4.28 | 0.09 | 99.93^{c} | |
| Lyso PC | 8.45 | 0.23 | 1.54 | 0.03 | 96.78 | |
| Ether PC | 3.02 | 0.17 | 3.56 | 0.01 | 99.48 | |
| SM | 1.98 | 0.01 | 2.80 | 0.12 | 97.38 | |
| SGL | 1.75 | 0.29 | 6.06 | 0.35 | 93.02 | |

^{a}Conversions of PC, lyso PC, ether PC, and SM were monitored in the positive ion mode, and the others were in the negative ion mode. Internal standards were added to unreacted and reacted mixtures. The amounts of remaining lipids were calculated by their relative abundance to the internal standards.
^{b}Labeling efficiency was monitored in the positive ion mode. No remaining lipid derivatives were observed.
^{c}Fragmented PS product and single-alkylated PA was included, while labeled PS and double-alkylated PA was the main product.

TABLE 2
Lipid standards used in this study.

| Lipid | | | Molecular |
|---|---|---|---|
| Class | Composition | Adduct | weight |
| PC | 15:0-18:1 (d7) | | 753.11 |
| PE | 15:0-18:1 (d7) | | 711.03 |
| PG | 15:0-18:1 (d7) | Na salt | 764.02 |
| PS | 15:0-18:1 (d7) | Na salt | 777.02 |
| PI | 15:0-18:1 (d7) | NH₄ salt | 847.13 |
| PA | 15:0-18:1 (d7) | Na salt | 689.93 |
| Lyso PC | 18:1 (d7) | | 528.72 |
| Ether | 16:0-18:1 | | 746.09 |
| PC | | | |
| SM | D18:1-18:1(d9) | | 738.12 |
| SGL | 18:1-18:1 | NH₄ salt | 880.23 |
| PC | 16:0-18:1 | | 760.08 |
| PE | 15:0-18:1 (d7) | | 744.03 |
| PG | 15:0-18:1 (d7) | Na salt | 797.02 |
| PS | 15:0-18:1 (d7) | Na salt | 810.02 |
| PI | 15:0-18:1 (d7) | NH₄ salt | 880.14 |
| PA | 15:0-18:1 (d7) | Na salt | 722.95 |

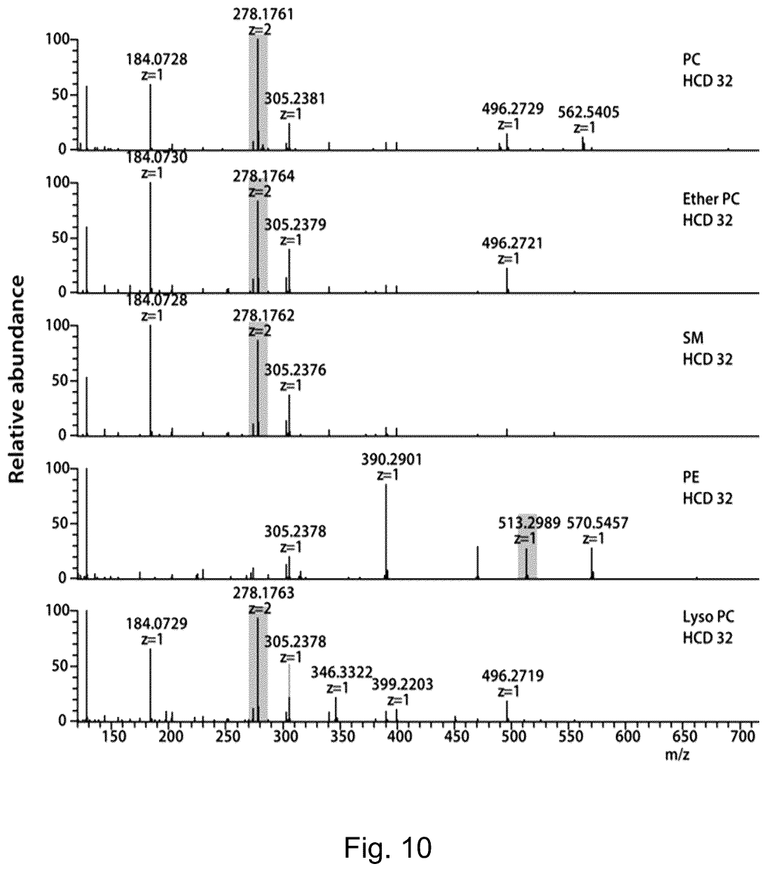

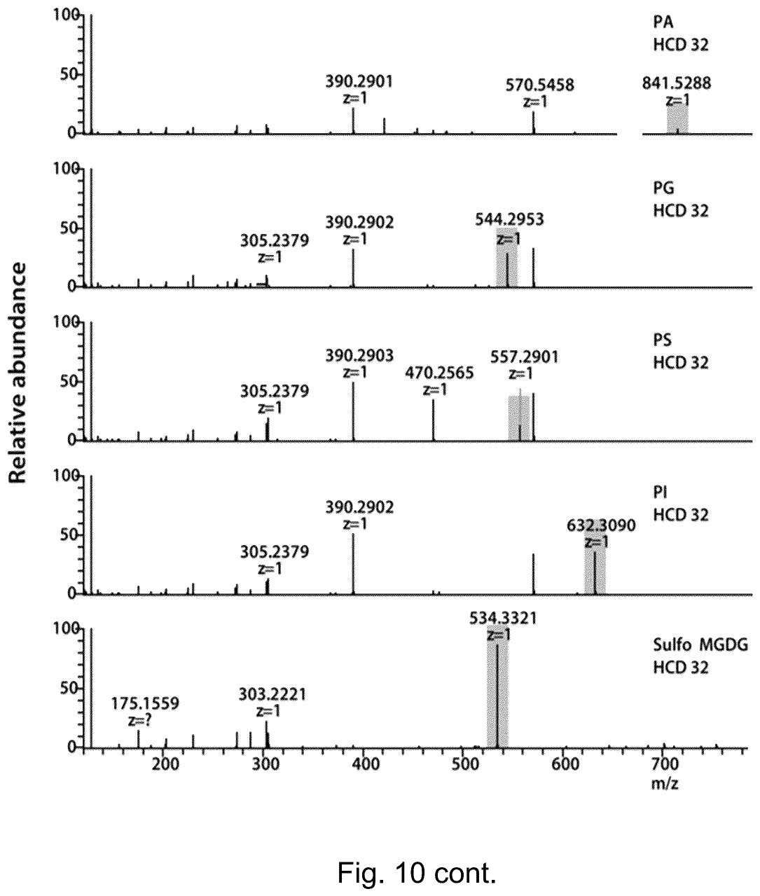

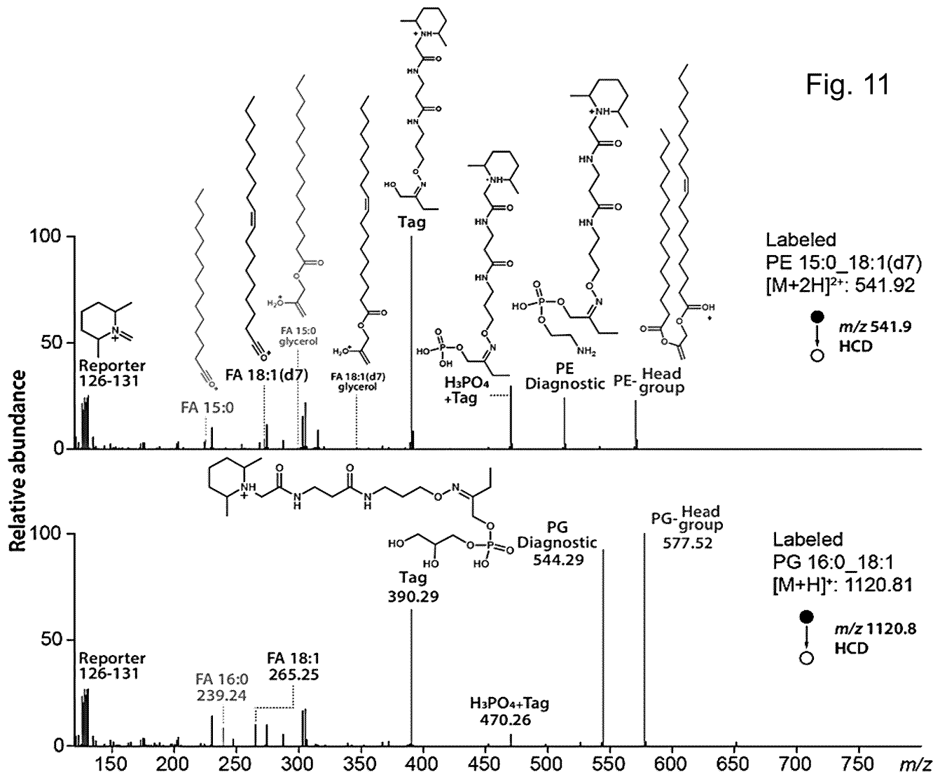

Next, the characteristics of the labeled lipids were investigated. During LC-MS analysis, the chromatography behavior of labeled lipids was similar to that of unlabeled lipids. Labeled lipids with longer aliphatic chains and lower unsaturated degrees tended to have longer retention times during C18 reversed-phase LC separation. In terms of MS characteristics, the amine groups of aminoxyTMT tags, which are favorably protonated under acidic mobile phases, caused PA and lipids with choline or ethanolamine to primarily exhibit doubly charged species. Labeled lipids displayed different fragmentation patterns from their original counterparts (FIG. 10). FIG. 9, panel b and FIG. 11 show representative MS2 spectra of labeled PE 15:0_18:1(d7) and PG 16:0_18:1. The peaks at m/z 126-131 represent the reporter ions generated from the aminoxyTMT tags used for multiplexed quantification. The peaks at m/z 513 and 544 were the fragments of the PE and PG head groups with oxime-formed aminoxyTMT, indicating the lipid classes of the precursor ions.

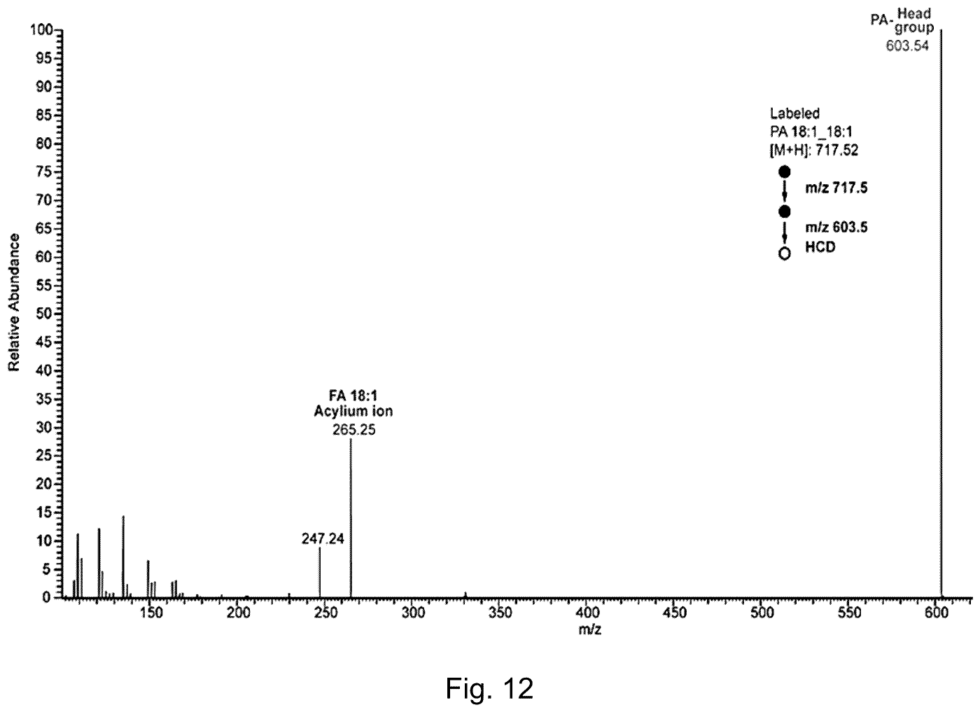

Lastly, the method provides information on acyl chains that can differentiate lipid isomers. The peaks located at 570 and 577 represent the neutral loss of polar head groups and can undergo MS3 fragmentation to elucidate fatty acid chains (FIG. 12). Additionally, single acyl chain-related fragments, which were not efficiently generated before labeling, appear in the MS2 level, eliminating the need for MS3 analysis for differentiating acyl chains. Using acylium ions ([RCO]⁺) or acyl chains with glycerol backbones ([RCOO+C₃H₅O+H]⁺), the PE species can be identified as PE 15:0_18:1(d7) and PG 34:1 as PG 16:0_18:1.

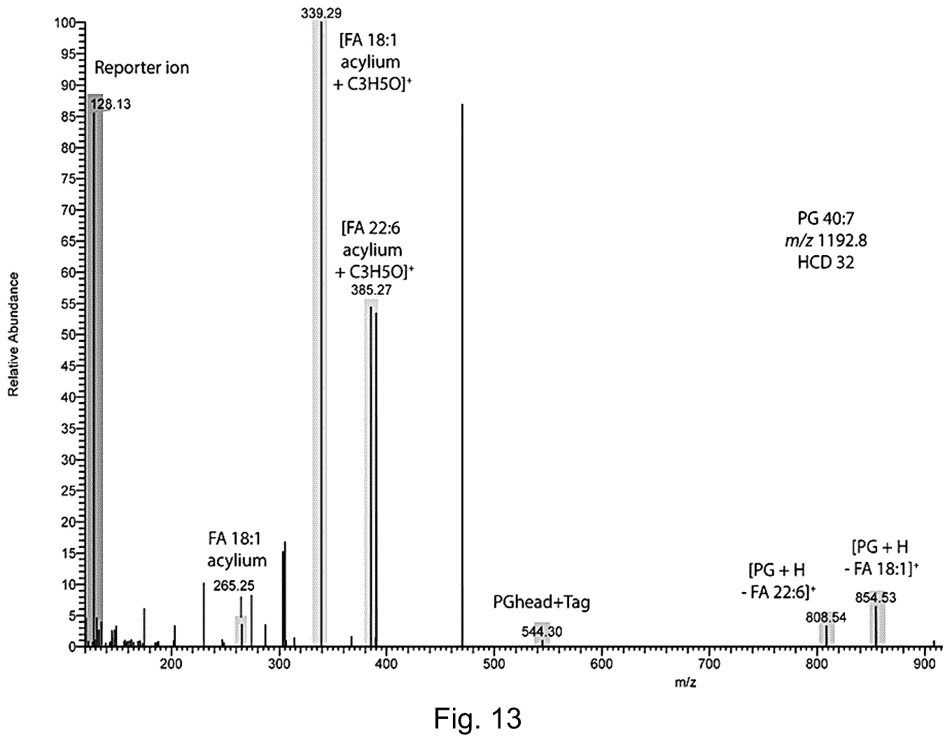

Neutral loss products ([M+H—RCOO]⁺) are also observed in some lipid species (FIG. 13). All fragmentation patterns and retention times of labeled lipids were considered to validate lipid identification, in addition to accurate mass matching.

### Example 4—Isobaric Labeling of Phospholipid Extract from Complex Samples

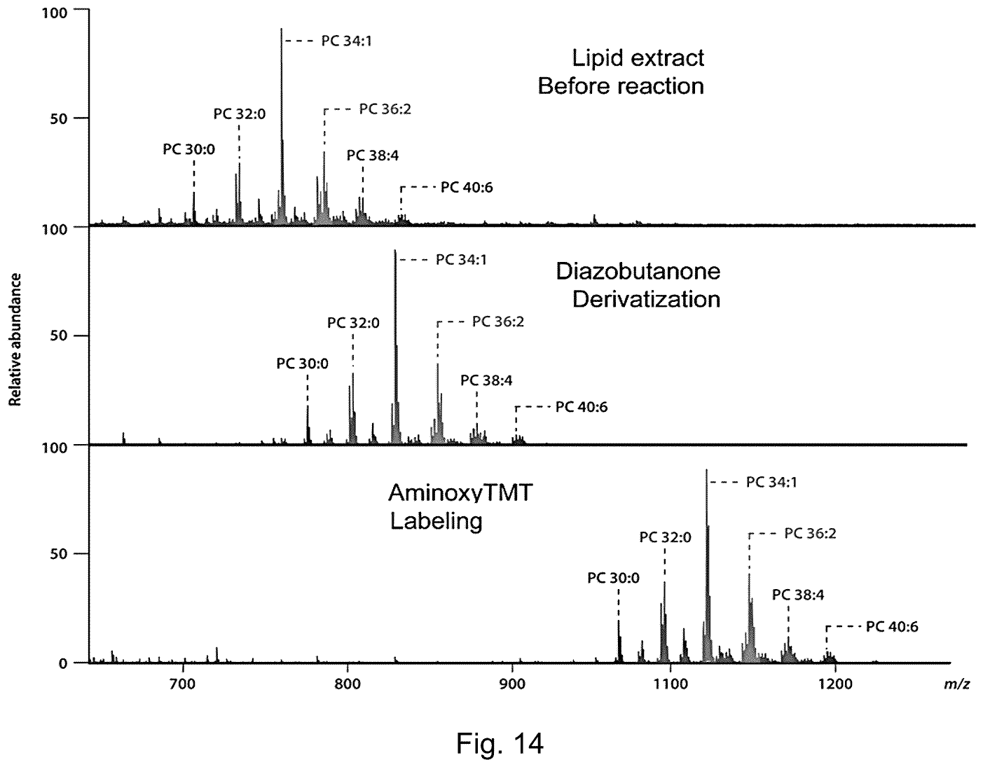

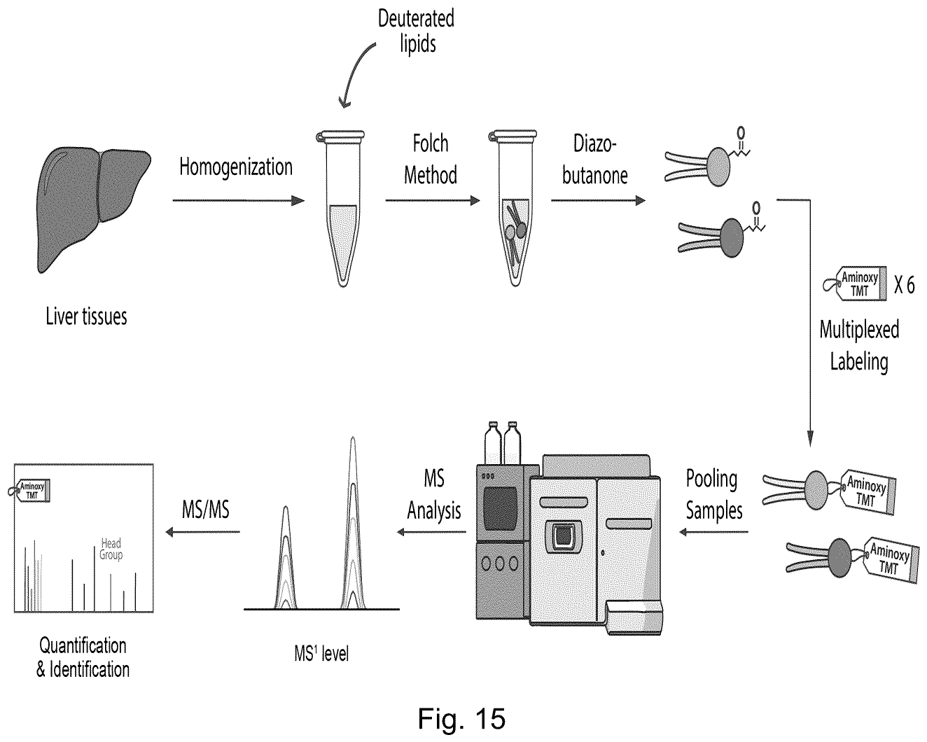

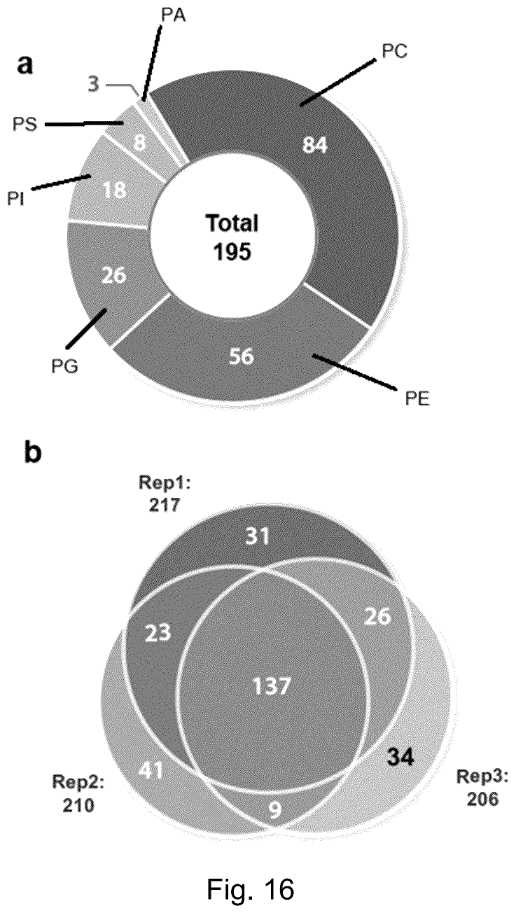

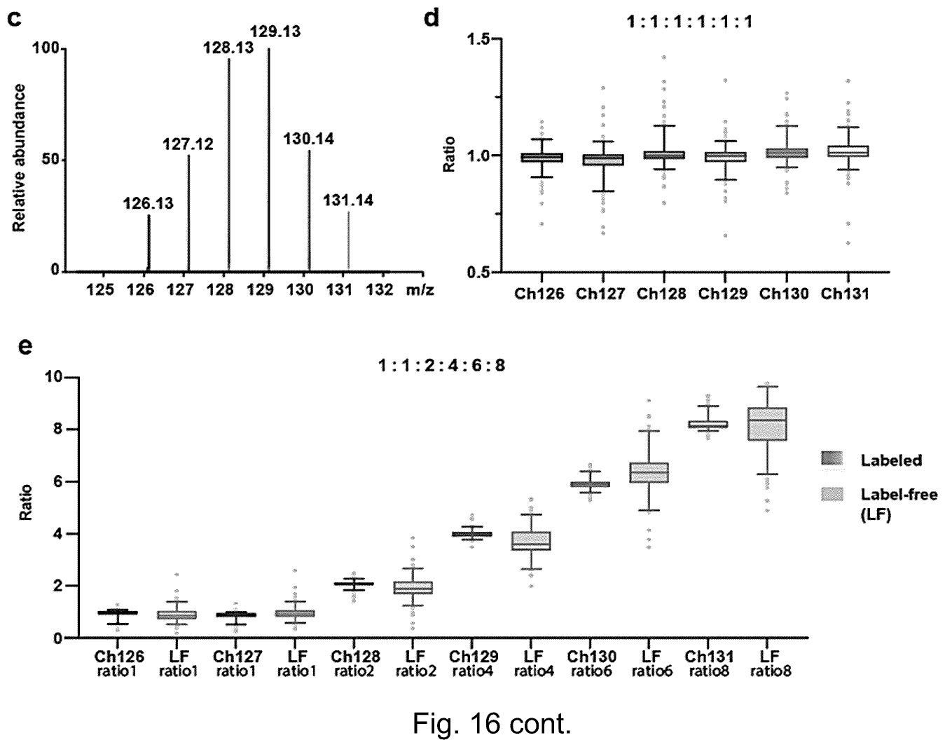

In mammalian cells, PC is the most abundant PL followed by PE and PI. Each PL class distributes differently among cell types and exerts its functions, and all lipid classes also collectively contribute to the homeostasis and maintenance of cellular environments²⁸. In this study, the method was applied to simultaneously profile the central PL classes (PC, PE, PI, PS, PG, and PA) in the human pancreatic cancer cell line PANC-1 and obese mouse liver tissues to demonstrate the applicability of the approach using complex biological samples (FIG. 14, FIG. 15, and FIG. 16).

From the full MS spectra (FIG. 14), it was observed that the lipid signal peaks from previous steps were completely consumed by each reaction, indicating that this two-step labeling procedure was highly compatible with complex systems. The labeled mixtures were then subjected to LC-MS/MS analysis in positive ion mode. The labeled PLs were identified by accurate mass matching to the database and the diagnostic ions for class identification using an in-house developed script in R program. Quantitative information was acquired by extracting the abundances of reporter ions from six aminoxyTMT channels. All MS2 fragmentation patterns, charges, and retention time were considered to validate their identification. In total, 304 PLs were identified in PANC-1 cells and 195 PLs were identified in mouse liver tissues using the modified LipidBlast database²⁹ (FIG. 16, panel a). In biological triplicate analysis (FIG. 16, panel b), approximately 80% of identifications were found in two of three replicates. Among them, PC accounted for 43% of identifications, PE for 29%, and PG for 13%. The lipid composition is comparable with previous label-free lipidomics analysis of mouse liver with nonalcoholic steatohepatitis^{30,31}, with higher percentages in PG, probably due to the enhanced detection sensitivity. These results demonstrate a high reproducibility and the ability to reliably profile the lipidome using the new method.

To evaluate the quantitative accuracy of the method for complex samples, a 6-plex diazobutanone-assisted isobaric labeling was conducted on six aliquots of PL extracts. The PL extracts were separately subjected to diazobutanone reaction and 6-plex aminoxyTMT labeling. The mixtures were then combined with molar ratios of 1:1:1:1:1:1 and 1:1:2:4:6:8 prior to MS analysis. Additionally, deuterated PL standards were added with a ratio of 1:2:4:4:2:1 to the mixtures to further assess the quantitative accuracy. The reporter ions of the deuterated PL standards showed an expected ratio of 1:2:4:4:2:1 (FIG. 16, panel c) without ratio distortion. All quantified PLs were plotted against each other (FIG. 16, panel d). The median coefficient of variation (CV) less than 10% for both theoretical ratios of 1:1:1:1:1:1 and 1:1:2:4:6:8 were obtained (FIG. 16, panel d and panel e), demonstrating excellent quantitative performance.

To compare the method with label-free approaches, a parallel label-free experiment was conducted using the same LC method in negative ion mode and quantified samples with a ratio of 1:1:2:4:6:8. Employing the same nano-LC gradient, label-free analysis required 6-10 times more instrument time, including blank washes, which might affect the consistency of subsequent samples. In terms of quantitative results, both label-free and isobaric labeling approaches were able to obtain the expected ratio of 1:1:2:4:6:8 (FIG. 16, panel e). However, the label-free approach displayed a median CV of 24%, which was twice that of the labeling method. Furthermore, the number of identified PLs dropped from 173 in LFᵣₐₜᵢₒ₈ to 105 in LFᵣₐₜᵢₒ₁. Although LC alignment allows for the quantification of PLs in analyses where PLs were not identified, misalignment and variations might be introduced, leading to inaccurate results. In contrast, since isobaric labeling quantifies lipids at MS2 levels, lipids in lower abundances in one sample will be isolated and quantified along with another sample with higher lipid abundance, ensuring accurate quantification of low-abundance lipids.

### Example 5—Relatively Quantify Phospholipids in Liver Tissues from Lean and Obese Mice

Obesity has become a serious health problem over the past few decades, contributing to an increased risk for various diseases, including non-alcoholic fatty liver disease, type 2 diabetes, and cancer. Aberrant lipid accumulation in the liver is a hallmark of these diseases³². Given the critical roles of lipids in biological systems, lipidomic analyses of liver tissues have the potential to not only decipher lipid functions in metabolic pathways but also identify potential biomarkers for disease diagnosis. Several studies have reported on the mechanisms by which lipids can cause the development of diseases. For example, a decreased PC/PE ratio has been found to be responsible for the progression of steatohepatitis and liver failure by affecting membrane integrity³³. Additionally, the aliphatic chains on high-density lipoprotein PLs are correlated with the capability of the efflux of cellular cholesterol³⁴.

Here, a 6-plex quantitative analysis was performed on liver tissues from three healthy, lean male mice and three insulin-resistant, obese male ob/ob mice. Equal amounts of liver tissues were collected and homogenized, and deuterated lipid standards were added to the mixtures before lipid extraction. Next, the extracted lipids underwent 6-plex diazobutanone-assisted isobaric labeling. PLs were identified by examining their exact masses and diagnostic ions, and the abundance of reporter ions was extracted from 6 channels. The data was normalized using deuterated lipid standards to correct for lipid recovery from lipid extraction.

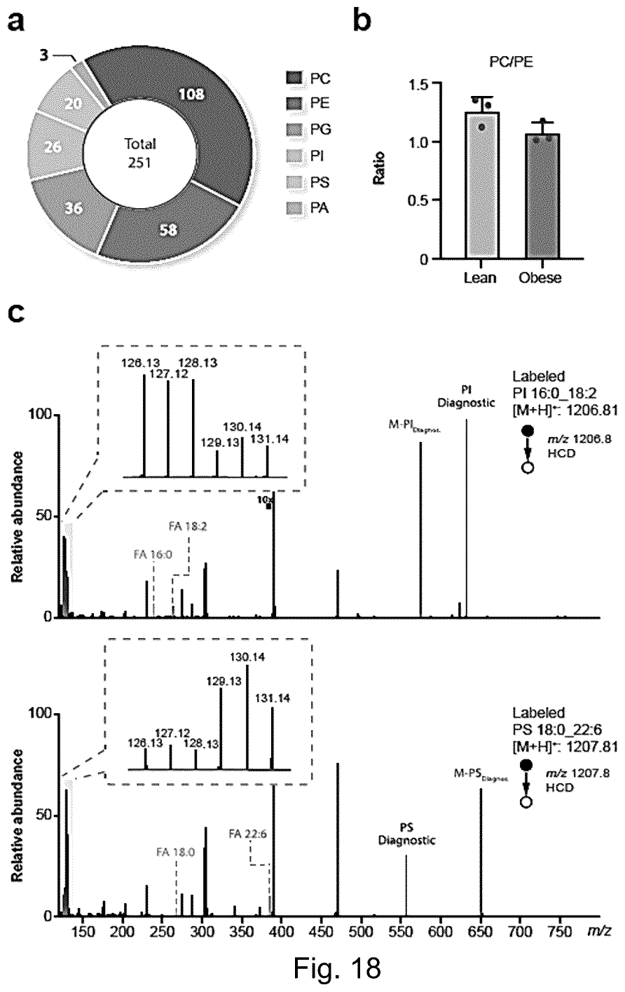

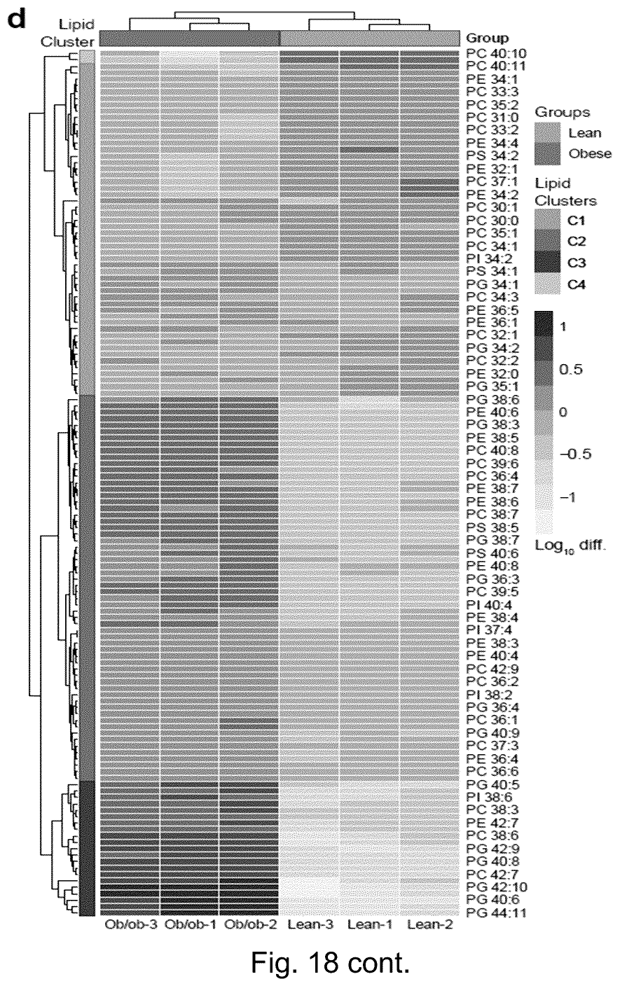

In total, 251 PL species were identified with acyl chain compositions in liver tissues from lean and obese mice (FIG. 18, panel a). These methods revealed 58 PLs and 100 PL molecular species enriched in obese mice, and 29 PLs and 24 PL molecular species enriched in lean mice (adjusted p-value<0.05). First, the progression indicator of hepatic disease, the PC/PE ratio^{33,38}, was examined using the intensity of reporter ions from PLs. A decreasing trend was observed in obese mice (FIG. 18, panel b), though a significant change was not apparent. This could be because the obese mice in the study exhibited insulin resistance but had not yet developed severe hepatic disease. Hierarchical clustering analyses (FIG. 18, panel d) showed remarkable reproducibility within groups and produced strong clustering of the two groups. Four main clusters of lipids can be visually identified, including one cluster enriched in the lean mice group, two clusters enriched in the obese mice group, and one cluster without obvious enrichment.

In these findings, phospholipids (PLs) with relatively long acyl chains (>38 carbon atoms) were upregulated (FIG. 18, panels c and d), while PLs with shorter acyl chains (<34 carbon atoms) and PC species with high degrees of unsaturation were diminished in obese mice. This trend was in agreement with the previous report and could be attributable to the elongase Elovl6^{35,36}, which catalyzes the extension of long-chain fatty acids and has been reported to play a pivotal role in obesity-induced insulin resistance. Similar patterns have been observed in mice with hepatic disease, implying that the elongation of phospholipid chains in obesity may affect liver function. In the analysis where acyl chains were resolved, PLs with polyunsaturated acyl chains, such as C20:4 and C22:6, exhibited a significant increase in obese mice. This could be due to the upregulated expression of desaturases, enzymes vital for the synthesis of polyunsaturated fatty acids. This observation parallels findings in the liver phospholipids of mice subjected to a high-fat diet³⁷.

Overall, these results suggest that obesity and possibly insulin resistance led to a severe disturbance in lipid homeostasis, and besides PC, many lipid species in other classes that are not commonly studied also displayed strong correlations with obesity. It is possible that the results from these lipidomic studies on mouse livers may not be representative of results from human livers with metabolic diseases. Therefore, further investigations are needed to explore the functions of these key identified lipids and to ensure their relevance to human studies. Nonetheless, these distinct PL profiles between lean and obese mouse livers increase the understanding of the relationship between lipids and diseases. The ability of this method to analyze a broad range of lipid classes will facilitate quantitative lipidomics in comprehensive and high-throughput manners.

### Example 6—Discussion

Lipidomic reports often provide conflicting results with regard to lipid compositions, likely due to the non-standardized lipidomic procedures and variability occurring in label-free workflows. In this study, a novel diazobutanone-assisted isobaric labeling strategy is presented that enables accurate multiplexed quantification and structure elucidation of phosphate and sulfate-containing lipids. The rationale for the design and optimization of diazobutanone reaction is described in detail, highlighting the advantages of using this chemical reagent, including its easy, mild, and MS-compatible sample preparation protocols, efficient conjugation of phosphodiesters, and compatibility with various functional groups under optimized reaction conditions. These features make the method broadly applicable to complex biological sample types. Additionally, the isobaric tagging strategy is fast and cost-effective, making it favorable for large-scale lipidomics.

Using this method, phospholipids extracted from healthy and obese mouse livers were analyzed and quantified in a high-throughput manner. The alterations of phospholipid expression levels from all major classes were observed in the obese mouse models, indicating the alteration of enzyme activity involved in lipid metabolism in obesity. For example, the significant up-regulation of phospholipids with relatively long carbon chains might result from fatty acid elongases (Elovl-5,6).

However, current quantitative studies mainly focus on fatty acids or certain phospholipid classes, but lack the ability to comprehensively interrogate the lipidome. It is envisioned that this new method can be applied to investigate multiple classes of lipids and their dynamic interplay occurring in various diseases, and facilitate the discovery of lipid biomarkers in different diseases and physiological states in a non-targeted fashion. The diazobutanone-assisted isobaric labeling strategy provides a strong starting point for lipidomics to leverage the benefits of isobaric labeling, including higher quantitative accuracy, reproducibility, fewer missing values, elimination of matrix effect, and the capability to conduct analysis of replicates or multiple test groups within the same experiment. Advanced strategies that involve isobaric mass tags can also be achieved, such as adding boosting channels to enhance the detection of target analytes or low-abundance lipids, or bridge channels to normalize the quantification across multiple sample sets. In summary, the diazobutanone-assisted isobaric labeling will drive the field of quantitative lipidomics to unprecedented higher throughput analysis with enhanced coverage and improved sensitivity.

### Example 7—Methods

Materials and Reagents. All Lipid standards were purchased from Avanti Polar Lipids (Alabaster, Al). Optima LC/MS grade acetic acid (AA), acetonitrile (ACN), ammonium formate, formic acid, isopropanol (IPA), Methanol (MeOH), and water were purchased from Fisher Scientific (Pittsburgh, PA). ACS grade chloroform, dichloromethane (DCM), ether, ethyl acetate (EA), ethyl propionate, 3,5-heptanedione, tetrafluoroboric acid diethyl ether complex (HBF₄·Et₂O), p-toluenesulfonyl azide (TsN₃), and triethylamine (NEt₃) were purchased from Sigma-Aldrich (St. Louis, MO). Phosphate buffered saline (PBS) was purchased from Crystalgen (Commack, NY). AminoxyTMTsixplex™ label reagent was purchased from Thermo Fisher Scientific (Waltham, MA). All reagents were used without additional purification.

Pancreatic Cancer Cell (PANC1) Culture. The commercially available pancreatic cancer cell line PANC-1 was obtained from American Type Culture Collection (ATCC, Manassas, VA, USA) and was maintained in DMEM: F12 (Hyclone, GE Healthcare Life Sciences, Logan, Utah, USA) containing 10% fetal bovine serum (FBS) (Gibco, Origin: Mexico) and 1% penicillin-streptomycin solution (Gibco, Life Technologies Corporation, Grand Island, NY, USA). Cells were cultured in a 37° C. moisture incubator filled with 5% CO₂. Cells were trypsinized at 70%-90% confluence using 0.25% trypsin EDTA solution (Gibco, Life Technologies Corporation, Grand Island, NY, USA). The cell suspension was centrifuged at 300 g for 5 minutes, and the medium was discarded. Cells were resuspended in phosphate-buffered saline (PBS) (Gibco, Life Technologies Europe B. V., Bleiswijk, The Netherlands) and washed twice with PBS, and stored at −80° C. freezers until use.

Obese mouse model and liver collection. Animal care and experimental procedures were performed in accordance with the guidelines and regulations of the Institutional Animal Care and Use Committees from the University of Wisconsin-Madison and William S. Middleton Memorial Veterans Affairs to meet acceptable standards of humane animal care. Mice were housed in facilities with a standard light-dark cycle and fed ad libitum. Breeders were fed Teklad 2919 (Envigo), and at weaning, mice were fed Teklad 2920× (Envigo) until transfer from the Biomedical Research Model Services breeding core to the Wisconsin Institute for Medical Research vivarium, at which time mice were fed Teklad 2018 (Envigo). Obese male C57BL/6N ob/ob mice at 13-14 weeks of age were generated by backcrossing the C57BL/6J ob allele (Jackson Laboratory Strain #000632) to C57BL/6NTac (Taconic) mice and breeding heterozygous C57BL/6N ob/+ mice to produce ob/ob mice. Mice were genotyped for the ob allele by PCR as previously described (Ellett J D et al. 2009). Lean male C57BL/6NTac mice at 16 weeks of age, generated in a breeding colony, were used as controls. The liver was isolated from non-fasted mice after euthanasia by carbon dioxide inhalation and flash-frozen in liquid nitrogen.

Sample preparation and PL extraction. Approximately 10 mg of liver tissue from lean and obese mice was weighed and dissolved in 0.35 mL PBS buffer. 3 μl of EquiSPLASH™ deuterated lipid standards (Avanti Lipids, Inc.) were added to the mixtures. The tissue was homogenized with a probe sonicator in an ice water bath at 50% power with pulse 12 s on and 12 s off for 12 cycles. The homogenates were then transferred to glass tubes for lipid extraction. Lipid extraction from cells or liver tissues was initiated by adding 1.5 mL methanol/chloroform (2:1 v/v) and vortexing for 15 min, followed by adding 0.6 mL chloroform and 0.6 mL H₂O. The mixture was vortexed for another 15 min and then centrifuged at 2000×g for 10 min to separate the liquid layers. The bottom layer was collected, and the extraction of the aqueous layer was repeated twice with 1 mL chloroform. The organic layers were combined and dried under a nitrogen stream. Subsequently, 1.8 mL hexane/methanol/ddH2O (1:1:0.1 v/v/v) was added for lipid extraction. The mixture was vortexed for 5 min and then centrifuged at 2000×g for 10 min. The bottom layer was collected to obtain the phospholipid extract, which was then dried under a nitrogen stream and stored at −20° C. until use.

Diazobutanone reaction of phospholipids and isobaric labeling. The synthesis of diazobutanone and the lipid estimation method are provided below. 5 μg of phospholipid (PL) standards or approximately 5 μg of PL extracts was dissolved in 42.5 μL of ethyl propionate. To this solution, 50 μL of a freshly prepared 0.48 mM tetrafluoroboric acid dimethyl ether complex in ethyl propionate was added, along with 7.5 μL of a 10:1 (v/v) solution of ethyl propionate and diazobutanone. The reaction proceeded at room temperature for 35 minutes, before quenching with 400 μL of 0.7% formic acid in isopropanol. The reaction mixture was subsequently dried under vacuum.

For 6-plex AminoxyTMT isobaric labeling, the optimized labeling protocol was modified according to the manufacturer's instructions (Thermo Fisher Scientific). In the case of lipid standards, the derivatized PL sample was labeled with 10 μg of aminoxyTMT in 20 μL of a solution containing AA/IPA/MeOH (0.1:10:90 v/v/v). For complex samples, the samples were labeled with 20 μg of aminoxyTMT (at a lipid-to-aminoxyTMT ratio of 1:4, accounting for potentially reactive impurities in complex samples) in 40 μL of the same solution. The reaction mixture was incubated at room temperature for 15 minutes and then dried under vacuum. Next, 20 or 40 μL of IPA/MeOH (1:9 v/v) was added, vortexed the mixture for 10 minutes, and dried it under vacuum. Subsequently, the mixture was extracted twice with 15 μL of 0.02% acetic acid in water and 100 μL of ethyl acetate. The collected organic layers were dried and stored at −80° C. until MS analysis.

MS analysis. Direct infusion and MS3 analysis of lipids were performed on Thermo Scientific Orbitrap Elite mass spectrometer (San Jose, CA) with an ESI source. For LC-MS analysis, the sample was analyzed using a binary nanoAcquity UPLC system (Waters, Milford, MA) coupled with a Q Exactive mass spectrometer (Thermo Scientific, San Jose, CA) to monitor the derivatives and labeled PLs. Labeled PLs were dissolved in a 30% phase B solution. The samples were loaded onto a self-fabricated microcapillary column packed with C18 beads (Waters Bridged Ethylene Hybrid, 1.7 μm, 130 Å, 101.3 μm×15 cm).

For mobile phases, phase A consisted of ACN:H₂O:MeOH:IPA (2:2:2:1) with 10 mM ammonium formate and 0.1% formic acid. Phase B consisted of IPA:ACN (9:1) with 10 mM ammonium formate and 0.1% formic acid. PLs were separated using a gradient elution of 5-60% B over 10 min and 60-95% B over 60 min at a flow rate of 300 nL/min.

For precursor MS scans, 440-1440 m/z were collected at a resolving power of 70 k (at 200 m/z) with an automatic gain control (AGC) target of 1×10⁶ and a maximum injection time of 200 ms. Data-dependent MS/MS analysis was performed with an inclusion list using HCD with 32% normalized collision energy at a resolving power of 17.5 k, and the top 15 precursors were selected for HCD analysis. The AGC, maximum injection time, resolution (at m/z 200), and lower mass limit for tandem mass scans were 1×10⁶, 500 ms, 17.5 k, and 120 m/z, respectively. Precursors were subjected to dynamic exclusion for 5 s.

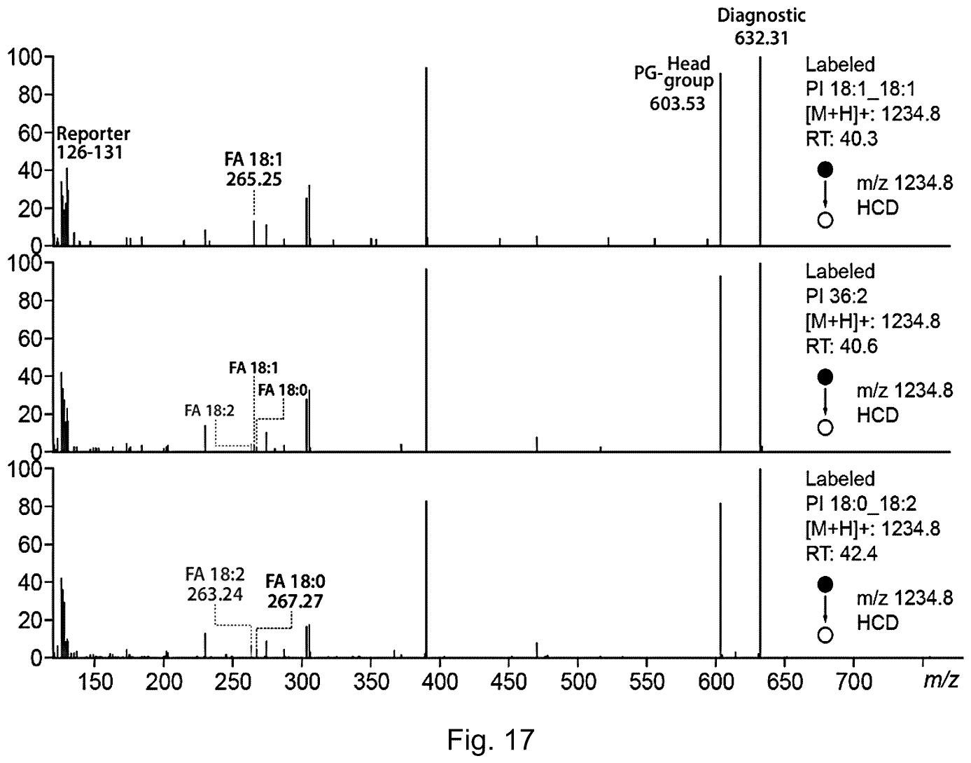

Data analysis. The list of fatty acids and the PL database were obtained and modified from LipidBlast to incorporate the mass increments resulting from the labeling. PL identification was based on retention time, precursor mass accuracy, and fragmentation patterns. Accurate mass tolerance for identification was set at 5 ppm for both MS full scans and MS/MS. Diagnostic ions were used for class identification, while [M+H—RCOO]⁺ was used for PG identification since diagnostic ion intensity for some PG species was low. Acyl chain elucidation was based on at least one acylium ion found in the spectra, and retention time was manually checked for correct assignment. The quantification of labeled PL with acyl chain information was performed based on the detection of acylium ion and reporter ions from the same spectra (FIG. 17). To identify lipids enriched in either group of mice, t-tests were conducted on the log-ratio per PL and PL species. FDR-adjusted p-values (<0.05) were calculated to account for multiple testing. The logged ratios of PL and PL species were subtracted by their logarithmic mean and used for hierarchical clustering analyses. Identification, quantification, and bioinformatics analyses, including hierarchical clustering, were carried out in the R statistical computing environment using R packages (dplyr, lubridate, magrittr, MSnbase, xcms, openxlsx, reshape2, and pheatmap). Label-free analysis was performed using LipidSearch software version 5.1 (Thermo Scientific).

Estimation of lipid amount. A common lipid extraction method, the Folch extraction, was employed, which typically achieves approximately a 90% recovery rate for phospholipids from biological samples. Consequently, deuterated lipids, added prior to lipid extraction, were used to estimate the lipid amounts. In mammalian cells, phosphatidylcholines (PCs) generally constitute about 50% of total phospholipid amounts. In this approach, the intensity ratios were calculated between deuterated PC and the top few PC peaks to estimate the lipid amounts in the complex samples. For instance, in direct fusion or MALDI analysis, when 3 μg of deuterated PC was added to the mixture, the total intensity of the peaks at 760, 786, and 810 m/z was 100 times the intensity of the deuterated PC standard. In such a case, it was estimated that the total amount of phospholipids in the mixture was 600 μg. For optimal labeling performance, a lipid concentration of 50 μg/mL is recommended for diazobutanone derivatization. Nevertheless, the quantitative performance was also evaluated at lower lipid concentrations, specifically 0.5, 0.05, and 0.005 μg/mL, and found that accurate ratios were still successfully obtained.

Synthesis of diazobutanone. 3,5-heptanedione (250 mg, 1.95 mmol) and TsN₃ (514 mg, 2.61 mmol) were dissolved in 4 mL ACN at 0° C. NEt₃ (326 μL, 2.34 mmol) was added to the cooled solution and the resulting reaction was stirred for 4 h. The reaction was filtered through Celite and concentrated in vacuo. The residue was purified by silica gel column chromatography (0%→20% EA in hexane) to afford 3,5-dione-4-diazoheptane (256 mg, 85%) as a yellow liquid. ¹H NMR (400 MHz, CDCl₃) δ 3.15-2.28 (m, 4H), 1.13-1.09 (m, 6H). ESI-MS: m/z calcd for C₇H₁₀N₂O₂; 154.07423 found 155.0819 (M+H)⁺.

3,5-dione-4-diazoheptane (40 mg, 0.26 mmol) was dissolved in 530 μL ether at 0° C. NaOH (530 μL, 3M) was slowly added to the reaction mixture and the resulting reaction was stirred at 0° C. for 3 h. The aqueous phase was extracted with ether (3×500 μL). The combined organic layer was dried over MgSO₄ and filtered. The filtrate was concentrated under reduced pressure (T=20° C., P≥250 mbar) to afford diazobutanone as a volatile yellow liquid (24 mg, 95%). ¹H NMR (400 MHz, CDCl₃) δ 5.22 (s, 1H), 2.89-2.04 (m, 2H), 1.09 (td, 3H). ESI-MS: m/z calcd for C₄H₆N₂O; 98.04801 found 99.0559 (M+H)⁺.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components, or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

### REFERENCES

1. Blanksby, S. J. & Mitchell, T. W. Advances in Mass Spectrometry for Lipidomics. Annu. Rev. Anal. Chem. 3, 433-465 (2010).
2. Meer, G. van, Voelker, D. R. & Feigenson, G. W. Membrane lipids: where they are and how they behave. Nat. Rev. Mol. Cell. Bio. 9, 112-124 (2008).
3. Tobias, F., Pathmasiri, K. C. & Cologna, S. M. Mass spectrometry imaging reveals ganglioside and ceramide localization patterns during cerebellar degeneration in the Npc1−/− mouse model. Anal. Bioanal. Chem. 411, 5659-5668 (2019).
4. Barrientos, R. C. & Zhang, Q. Recent advances in the mass spectrometric analysis of glycosphingolipidome—A review. Anal. Chim. Acta. 1132, 134-155 (2020).
5. Fu, S. et al. Aberrant lipid metabolism disrupts calcium homeostasis causing liver endoplasmic reticulum stress in obesity. Nature 473, 528-531 (2011).
6. Fadeel, B. & Xue, D. The ins and outs of phospholipid asymmetry in the plasma membrane: roles in health and disease. Crit. Rev. Biochem. Mol. 44, 264-277 (2009).
7. Salita, T., Rustam, Y. H., Mouradov, D., Sieber, O. M. & Reid, G. E. Reprogrammed Lipid Metabolism and the Lipid-Associated Hallmarks of Colorectal Cancer. Cancers 14, 3714 (2022).
8. Han, X. The emerging role of lipidomics in prediction of diseases. Nat. Rev. Endocrinol. 18, 335-336 (2022).
9. Danne-Rasche, N., Coman, C. & Ahrends, R. Nano-LC/NSI MS Refines Lipidomics by Enhancing Lipid Coverage, Measurement Sensitivity, and Linear Dynamic Range. Anal. Chem. 90, 8093-8101 (2018).
10. Unsihuay, D. et al. Imaging and Analysis of Isomeric Unsaturated Lipids through Online Photochemical Derivatization of Carbon-Carbon Double Bonds**. Angew. Chem. Int. Ed. 60, 7559-7563 (2021).
11. Randolph, C. E., Blanksby, S. J. & McLuckey, S. A. Toward Complete Structure Elucidation of Glycerophospholipids in the Gas Phase through Charge Inversion Ion/Ion Chemistry. Anal. Chem. 92, 1219-1227 (2020).
12. Ma, X. et al. Identification and quantitation of lipid C═C location isomers: A shotgun lipidomics approach enabled by photochemical reaction. Proc. National. Acad. Sci. 113, 2573-2578 (2016).
13. Jung, H. R. et al. High throughput quantitative molecular lipidomics. Biochim. Biophys. Acta 1811, 925-934 (2011).
14. Leng, J. et al. A highly sensitive isotope-coded derivatization method and its application for the mass spectrometric analysis of analytes containing the carboxyl group. Anal. Chim. Acta. 758, 114-121 (2013).
15. Sivanich, M. K., Gu, T., Tabang, D. N. & Li, L. Recent advances in isobaric labeling and applications in quantitative proteomics. Proteomics 22, 2100256 (2022).
16. Thompson, A. et al. Tandem Mass Tags: A Novel Quantification Strategy for Comparative Analysis of Complex Protein Mixtures by MS/MS. Anal. Chem. 75, 1895-1904 (2003).
17. Xiang, F., Ye, H., Chen, R. B., Fu, Q. & Li, L. J. N,N-Dimethyl Leucines as Novel Isobaric Tandem Mass Tags for Quantitative Proteomics and Peptidomics. Anal. Chem. 82, 2817-2825 (2010).
18. Hahne, H. et al. Carbonyl-Reactive Tandem Mass Tags for the Proteome-Wide Quantification of N-Linked Glycans. Anal. Chem. 84, 3716-3724 (2012).
19. Gu, T.-J., Feng, Y., Wang, D. & Li, L. Simultaneous multiplexed quantification and C═C localization of fatty acids with LC-MS/MS using isobaric multiplex reagents for carbonyl-containing compound (SUGAR) tags and C═C epoxidation. Anal. Chim. Acta. 1225, 340215 (2022).
20. Yang, T. et al. Lipid Mass Tags via Aziridination for Probing Unsaturated Lipid Isomers and Accurate Relative Quantification**. Angew. Chem. Int. Ed. 61, e202207098 (2022).
21. Laayoun, A. et al. Aryldiazomethanes for Universal Labeling of Nucleic Acids and Analysis on DNA Chips. Bioconjugate Chem. 14, 1298-1306 (2003).
22. Bourget, C. et al. Biotin-phenyldiazomethane conjugates as labeling reagents at phosphate in mono and polynucleotides. Bioorgan. Med. Chem. 0 13, 1453-1461 (2005).
23. Furuta, T. et al. Versatile Synthesis of Head Group Functionalized Phospholipids via Oxime Bond Formation. Org. Lett. 10, 4847-4850 (2008).
24. Mix, K. A., Aronoff, M. R. & Raines, R. T. Diazo Compounds: Versatile Tools for Chemical Biology. Acs Chem. Biol. 11, 3233-3244 (2016).
25. Takahashi, T. & Suzuki, T. Role of sulfatide in normal and pathological cells and tissues. J. Lipid. Res. 53, 1437-1450 (2012).
26. Wasslen, K. V., Canez, C. R., Lee, H., Manthorpe, J. M. & Smith, J. C. Trimethylation Enhancement Using Diazomethane (TrEnDi) II: Rapid In-Solution Concomitant Quaternization of Glycerophospholipid Amino Groups and Methylation of Phosphate Groups via Reaction with Diazomethane Significantly Enhances Sensitivity in Mass Spectrometry Analyses via a Fixed, Permanent Positive Charge. Anal. Chem. 86, 9523-9532 (2014).
27. Wei, F. et al. Rapid profiling and quantification of phospholipid molecular species in human plasma based on chemical derivatization coupled with electrospray ionization tandem mass spectrometry. Anal. Chim. Acta. 1024, 101-111 (2018).
28. Neumann, E. K., Comi, T. J., Rubakhin, S. S. & Sweedler, J. V. Lipid Heterogeneity between Astrocytes and Neurons Revealed by Single-Cell MALDI-MS Combined with Immunocytochemical Classification. Angew. Chem. Int. Ed. 131, 5971-5975 (2019).
29. Kind, T. et al. LipidBlast in silico tandem mass spectrometry database for lipid identification. Nat. Methods 10, 755-758 (2013).
30. Saito, K. et al. Characterization of hepatic lipid profiles in a mouse model with nonalcoholic steatohepatitis and subsequent fibrosis. Sci. Rep. 5, 12466 (2015).
31. Peng, H. et al. Offspring NAFLD liver phospholipid profiles are differentially programmed by maternal high-fat diet and maternal one carbon supplement. J Nutritional Biochem 111, 109187 (2023).
32. Vucenik, I. & Stains, J. P. Obesity and cancer risk: evidence, mechanisms, and recommendations. Ann. Ny. Acad. Sci 1271, 37-43 (2012).
33. Arendt, B. M. et al. Nonalcoholic fatty liver disease is associated with lower hepatic and erythrocyte ratios of phosphatidylcholine to phosphatidylethanolamine. Appl. Physiol. Nutr. Metab. 38, 334-340 (2013).
34. Koekemoer, A. L. et al. Large-Scale Analysis of Determinants, Stability, and Heritability of High-Density Lipoprotein Cholesterol Efflux Capacity. Arterioscler Thromb. Vasc. Biol. 37, 1956-1962 (2017).
35. Nam, M. et al. Lipidomic Profiling of Liver Tissue from Obesity-Prone and Obesity-Resistant Mice Fed a High Fat Diet. Sci. Rep. 5, 16984 (2015).
36. Matsuzaka, T. et al. Crucial role of a long-chain fatty acid elongase, Elovl6, in obesity-induced insulin resistance. Nat. Med. 13, 1193-1202 (2007).
37. Scheja, L. et al. Liver TAG Transiently Decreases While PL n-3 and n-6 Fatty Acids are Persistently Elevated in Insulin Resistant Mice. Lipids 43, 1039-1051 (2008).
38. Jordy, A. B. et al. Analysis of the liver lipidome reveals insights into the protective effect of exercise on high-fat diet-induced hepatosteatosis in mice. Am. J. Physiol-endoc. M. 308, E778-E791 (2015).

1. A method for functionalizing a biomolecule comprising a phosphate group or sulfate group, the method comprising:
a) contacting the biomolecule with a linker comprising a first region having a diazo group and a second region having a ketone group, thereby reacting the first region of the linker with the phosphate group or sulfate group of the biomolecule and generating a conjugated biomolecule; and
b) contacting the conjugated biomolecule with a tagging reagent, thereby reacting the second region of the linker to the tagging reagent and generating a functionalized biomolecule.

2. The method of claim 1, wherein the linker has the following formula: 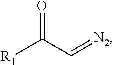
wherein R1 is hydrogen or is an alkyl group or an aromatic ring having 6 carbon atoms or less.

3. The method of claim 1, wherein R1 is an alkyl group having 1 to 3 carbon atoms.

4. The method of claim 1, wherein the linker has the following formula:

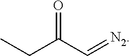

5. The method of claim 1, wherein the linker has the following formula:

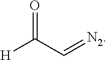

6. The method of claim 1, wherein the linker is volatile and has a boiling point less than 100° C.

7. The method of claim 1, wherein reacting the first region of the linker with the phosphate group or sulfate group of the biomolecule forms an alkylated phosphate or alkylated sulfated group.

8. The method of claim 1, wherein the tagging reagent comprises an aminooxy group able to form a reaction with the ketone group of the linker via oxime bond formation.

9. The method of claim 1, wherein the tagging reagent comprises at least one atom that is isotopically labeled.

10. The method of claim 1, wherein the tagging reagent comprises a reporter group, a balance group, and a carbonyl-reactive group, wherein one or more atoms in the reporter group, balance group, or both, are isotopically heavy versions of the atom.

11. The method of claim 1 further comprising performing mass spectrometry analysis on the labelled biomolecule.

12. The method of claim 11, wherein the mass spectrometry analysis comprises fragmenting the labelled biomolecule.

13. The method of claim 1, wherein the biomolecule is a phosphate-containing lipid or metabolite or a sulfate-containing lipid or metabolite.

14. The method of claim 1 further comprising generating the linker, wherein generating the linker comprises the steps of functionalizing a dione to contain a diazo functional group and treating the functionalized dione with a basic solution, thereby generating the linker comprising the first region having the diazo group and the second region having the ketone group, wherein generating the linker has a reaction yield of 75% or greater.

15. The method of claim 1 wherein reacting the first region of the linker with the phosphate group or sulfate group of the biomolecule further comprises removing excess amount of the linker by vacuum.

16. The method of claim 1 wherein reacting the first region of the linker with the phosphate group or sulfate group of the biomolecule has a derivatization efficiency of 93% or greater.

17. The method of claim 1 wherein generating the labelled biomolecule by reacting the second region of the linker to the tagging reagent has a labeling efficiency of 99% or greater.

18. The method of claim 1 comprising:
a) providing two or more samples, wherein each sample comprises an amount of the biomolecule;
b) contacting the biomolecule in the two or more samples with the linker thereby generating a conjugated biomolecule in each of the two or more samples;
c) labeling the conjugated biomolecule in the two or more samples with two or more tagging reagents, wherein each of the two or more samples is labeled with a different tagging reagent,
wherein each of the different tagging reagents comprises a reporter group and a balance group, wherein one or more atoms in the reporter group, balance group, or both, are isotopically heavy versions of the atom,
wherein the reporter group of each of the different tagging reagents has a different mass due to differently isotopically labeled atoms in each reporter group, and the balance group of each of the different tagging reagents has a different mass due to the differently isotopically labeled atoms in each balance group, and the total mass of each of the different tagging reagent is the same;
d) fragmenting the labeled biomolecules in each of the two or more samples and analyzing fragments of the labeled biomolecules from each of the two or more samples.

19. The method of claim 18, wherein each of the different tagging reagents comprise an aminooxy group able to form a reaction with the ketone group of the linker.

20. The method of claim 18 further comprising quantifying reporter ion intensities of the labeled biomolecule in each of the two or more samples.

21. A method for multiplexed analysis of a target biomolecule comprising a phosphate group or sulfate group, the method comprising the steps of:
a) providing two or more samples, wherein each sample comprises an amount of the target biomolecule;
b) contacting the target biomolecule in the two or more samples with the linker thereby generating a conjugated biomolecule in each of the two or more samples;
c) labeling the conjugated biomolecule in the two or more samples with two or more tagging reagents, wherein each of the two or more samples is labeled with a different tagging reagent,
wherein each of the different tagging reagents comprises a reporter group and a balance group, wherein one or more atoms in the reporter group, balance group, or both, are isotopically heavy versions of the atom,
wherein the reporter group of each of the different tagging reagents has a different mass due to differently isotopically labeled atoms in each reporter group, and the balance group of each of the different tagging reagents has a different mass due to the differently isotopically labeled atoms in each balance group, and the total mass of each of the different tagging reagent is the same;
d) fragmenting the labeled biomolecules in each of the two or more samples and analyzing fragments of the labeled biomolecules from each of the two or more samples.

22. The method of claim 21 further comprising quantifying amounts of the labeled biomolecule in each sample.

23. The method of claim 21, wherein the tagging reagent comprises a fluorescent agent.

24. A kit comprising a linker and two or more tagging reagents,
wherein the linker has the following formula: 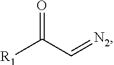
wherein R1 is an alkyl group or an aromatic ring having 1 to 6 carbon atoms,
wherein each tagging reagent comprises a reporter group, a balance group, and a carbonyl-reactive group wherein one or more atoms in the reporter group, balance group, or both, are isotopically heavy versions of the atom,
wherein the reporter group of each tagging reagent has a mass different than the reporter groups of the other tagging reagents, the balance group of each tagging reagent has a mass different than the balance groups of other tagging reagents, and the total mass of the reporter group plus the balance group for each tagging reagent is the same.

25. The kit of claim 24, wherein each reagent comprises an aminooxy group able to form a reaction with a ketone group in a conjugated biomolecule to be labeled.

26. The kit of claim 24, wherein R1 is an alkyl group having 1 to 3 carbon atoms.

27. The kit of claim 24, wherein the linker has the following formula:

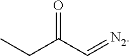

28. The kit of claim 24, wherein the linker has the following formula:

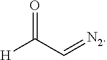

29. A compound comprising the formula: 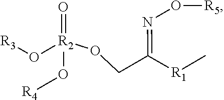
wherein,
R1 is an alkyl group or an aromatic ring having 1 to 6 carbon atoms;
R2 is P or S;
R3 is an aliphatic part having the formula: 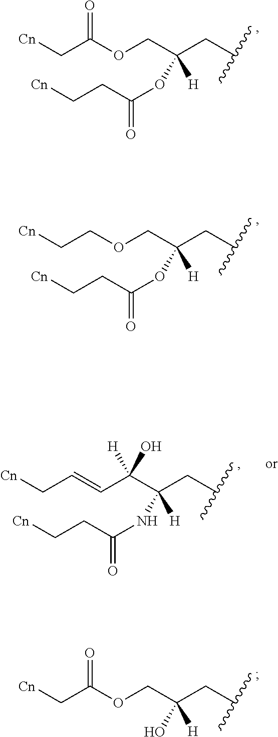
R4 is H or a head group having the formula: 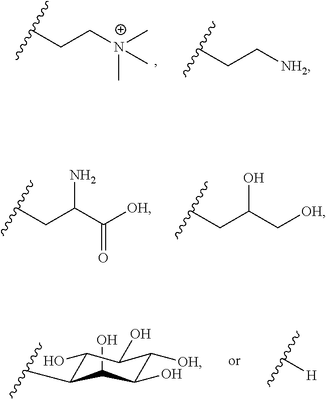
wherein when R2 is 8 R4═H; and
and R5 is a tagging reagent.

30. The compound of claim 29, wherein R1 is —CH₂— and R2 is —CH—.
